(19) United States
(12) Patent Application Publication
(10) Pub. No.: US 2014/0148364 A1
(43) Pub. Date: May 29, 2014

(54) MULTIPLEXED ANCHOR SCANNING PARALLEL END TAG SEQUENCING

(71) Applicant: Chaouki Miled, Antony (FR)

(72) Inventor: Chaouki Miled, Antony (FR)

(21) Appl. No.: 13/976,921

(22) PCT Filed: Dec. 5, 2011
(86) PCT No.: PCT/FR11/00634

§ 371 Date: Oct. 30, 2013

(30) Foreign Application Priority Data
Dec. 13, 2010 (FR) 1004835

(51) Int. Cl. C12Q 1/68

(52) U.S. Cl. CPC C12Q 1/6806
USPC 506/17; 506/16; 506/26

(57) ABSTRACT

The present invention relates to a novel method and to a kit for preparing nucleic-acid libraries, in particular for high-throughput sequencing. Said method is useful for simultaneously preparing multiple nucleic-acid libraries for sequencing, each library being characterized by a specific sequence of barcodes. In other words, instead of preparing, in parallel, a plurality of libraries that will be barcoded, the method of the invention enables the simultaneous preparation of a plurality of barcoded libraries by carrying out the step of preparing a single library. The inventor provides a means for inserting the barcodes at the beginning of the method for preparing the library. The libraries provided with the method described herein can be used for any high-throughput sequencing platform, such as the 454 Genome Sequencer, the Illumina Genome Analyzer, or the SoLiD platform.

### FIELD OF THE INVENTION

The present invention relates to the field of DNA sequencing and more particularly to the field of sequencing library preparation.

### BACKGROUND OF THE INVENTION

Since the early 1990s, DNA sequence production has almost exclusively been carried out with sequencing technology based on the Sanger biochemistry. A new generation of sequencing technologies has been recently developed. These high-throughput sequencing technologies are also known as “next generation DNA sequencing” or NGS technologies. Various implementations of these technologies have been performed in a commercial product such as the 454 Genome Sequencer (Roche Applied Science), the Illumina Genome Analyzer (Illumina, Inc.) or the SOLiD™ platform (Applied Biosystems). All these platforms rely on sequencing by synthesis, a serial extension of primed templates driven either by a polymerase or by a ligase. Thus, the sequencing process consists of alternating cycles of enzyme-driven extension and data acquisition.

Sequencing libraries used with NGS technologies are usually prepared by random fragmentation of DNA followed by in vitro ligation of common adaptor sequence. Sequencing features are generated by amplification with common PCR primers and are then immobilized or attached to a solid surface or support. NGS platforms are of low costs as they are able to simultaneously decode a two-dimensional array bearing millions of distinct sequencing features. Indeed, all immobilized array features can be enzymatically manipulated by a single reagent volume and the cost of this reagent is amortized over the full set of sequencing features. In order to maximize the sequencing capacity in a single run and thus to reduce the cost of sequencing per raw base, multiplexing methods have been developed. Barcoded adaptators are ligated to each library and several barcoded libraries can be pooled to be sequenced in a single run. The problem is that preparing several libraries is time and money consuming. Indeed, a library preparation requires about one week of work.

Next-generation sequencing platforms are high throughputs platforms of low costs but are limited by short read lengths. A solution to this limitation is the paired-end tag (PET) sequencing in which short and paired tags are extracted from the ends of long DNA fragments and are covalently linked as ditag constructs for high-throughput sequencing and mapping to reference genomes (Fullwood et al. 2009). PET strategy is the only technique adapted to NGS technologies and allowing the detection of deletion, inversion, tandem duplication, insertion and translocation which are currently associated with human diseases. Consequently, there is a strong demand for efficient, low-cost methods for the preparation of sequencing library for next generation sequencing technologies and in particular for the preparation of barcoded PET library.

### SUMMARY OF THE INVENTION

The present invention, called Multiplexed Anchor Scanning Parallel End Tag Sequencing (MASPET sequencing), relates to a method for preparing a nucleic acid library, preferably a DNA library, to be sequenced, wherein said method comprises:

a) providing a set or a plurality of sets of linear nucleic acid molecules, each nucleic acid molecule comprising in its circularized form and in the 5′ to 3′ direction
a sequence of interest to be entirely or partially sequenced
a reverse priming site,
a forward priming site,
a barcode sequence specific of each set of nucleic acid molecules, which is located between the forward priming site and the sequence of interest or between the sequence of interest and the reverse priming site, and
two recognition sites for two different restriction enzymes:
   a first recognition site for a first restriction enzyme which is located between the barcode sequence and the sequence of interest, and
   a second recognition site for a second restriction enzyme which is located between the reverse priming site and the forward priming site;

b) circularizing said linear nucleic acid molecules by intra-molecular ligation;

c) digesting circularized nucleic acid molecules obtained from step b) with the first restriction enzyme;

d) cleaving digested nucleic acid molecules obtained from step c) in the sequence of interest; and

e) circularizing said cleaved nucleic acid molecules obtained from step d) by intra-molecular ligation,

thereby providing circularized nucleic acid molecules comprising, in the 5′ to 3′ direction, a truncated sequence of interest, a reverse priming site, a forward priming site and a barcode sequence which is between the forward priming site and the sequence of interest or between the sequence of interest and the reverse priming site.

In a first embodiment, the linear nucleic acid molecules provided in step a) comprise in their circularized form and in the 5′ to 3′ direction, a sequence of interest, a reverse priming site, a forward priming site and a barcode sequence located between the forward priming site and the sequence of interest.

In a particular embodiment, the linear nucleic acid molecules provided in step a) further comprise a recognition site for a third restriction enzyme located upstream to the reverse priming site, said enzyme having a cleavage site upstream to said recognition site in the sequence of interest, and wherein the cleavage of step d) of claim 1 is achieved by digestion with said third restriction enzyme.

In a further particular embodiment, the nucleic acid molecules provided in step a) further comprise a recognition site for a fourth restriction enzyme located upstream to the reverse priming site and wherein the method further comprises after step e):

f) digesting circularized nucleic acid molecules obtained from step e) with the fourth restriction enzyme having a cleavage site located between the reverse priming site and the sequence of interest in said circularized molecules of step e); and

g) cleaving digested nucleic acid molecules obtained from step f) in the sequence of interest, thereby providing linear nucleic acid molecules comprising from 5′ end to 3′ end, a reverse priming site, a forward priming site, a barcode sequence and a sequence of interest truncated in 3′; and

h) optionally, circularizing nucleic acid molecules obtained from step g) by intra-molecular ligation.

Alternatively, the linear nucleic acid molecules provided in step a) comprise from 5′ end to 3′ end, a reverse priming site, a forward priming site, a barcode sequence and a sequence of interest, and the method further comprises, after step a) and before step b), the step of cleaving linear nucleic acid molecules provided in step a), thereby providing linear nucleic acid molecules comprising from 5′ end to 3′ end, a reverse priming site, a forward priming site, a barcode sequence and a sequence of interest truncated in 3′.

In a second embodiment, the linear nucleic acid molecules provided in step a) comprise in their circularized form and in the 5′ to 3′ direction, a reverse priming site, a forward priming site, a sequence of interest and a barcode sequence located between the sequence of interest and the reverse priming site.

Preferably, the linear nucleic acid molecules provided in step a) further comprise a recognition site for a third restriction enzyme located downstream to the forward priming site, said enzyme having a cleavage site downstream to said recognition site in the sequence of interest, and wherein the cleavage of step d) of claim 1 is achieved by digestion with said third restriction enzyme.

Alternatively, nucleic acid molecules provided in step a) further comprise a recognition site for a fourth restriction enzyme located downstream to the forward priming site, and wherein the method further comprises after step e):

f) digesting circularized nucleic acid molecules obtained from step e) with the fourth restriction enzyme having a cleavage site located between the forward priming site and the sequence of interest in said circularized molecules; and

g) cleaving digested nucleic acid molecules obtained from step f) in the sequence of interest, thereby providing linear nucleic acid molecules comprising from 5′ end to 3′ end, a sequence of interest truncated in 5′, a barcode sequence, a reverse priming site and a forward priming site; and

h) optionally circularizing nucleic acid molecules obtained from step g) by intra-molecular ligation.

In another alternative, the linear nucleic acid molecules provided in step a) comprise from 5′ end to 3′ end, a sequence of interest, a barcode sequence, a reverse priming site and a forward priming site, and the method further comprises, after step a) and before step b), the step of cleaving linear nucleic acid molecules provided in step a), thereby providing linear nucleic acid molecules comprising from 5′ end to 3′ end, a sequence of interest truncated in 5′, a barcode sequence, a reverse priming site and a forward priming site.

Preferably, the nucleic acid molecules provided in step a) comprise a binding site for a first member of an affinity binding pair or is attached to a first member of an affinity binding pair, and the method further comprises one or several steps of binding nucleic acid molecules on a solid support through the interaction between the first member of an affinity binding pair attached to said nucleic acid molecules and second members of said affinity binding pair linked to the solid support, in particular before a circularizing step.

Preferably, the method further comprises the step of digesting circularized nucleic acid molecules with the second restriction enzyme, thereby providing linear nucleic acid molecules comprising from 5′ end to 3′ end, a forward priming site, a truncated sequence of interest, a reverse priming site and a barcode sequence which is between the forward priming site and the sequence of interest or between the sequence of interest and the reverse priming site. More preferably, the method further comprises the step of amplifying barcode sequences and truncated sequences of interest from said linear nucleic acid molecules by using a pair of primers hybridizing on reverse and forward priming site.

Preferably, the steps of cleaving nucleic acid molecules are performed by using a sequence independent technique of cleavage, preferably by sonication.

Preferably, the linear nucleic acid molecules provided in step a) further comprise a universal priming site upstream or downstream to the sequence of interest.

Preferably, the linear nucleic acid molecules provided in step a) further comprise a curvature module which is located, in their circularized form, between the reverse priming site and the forward priming site, and which is attached to the first member of an affinity binding pair, preferably a curvature module comprising or consisting of the sequence selected from the group consisting of sequences of SEQ ID No. 1 and SEQ ID No. 2.

The present invention relates to a nucleic acid library obtained from the method disclosed herein or any intermediate product thereof.

The present invention relates to a kit comprising at least one first forward primer comprising, from its 5′ end to its 3′ end,
a reverse priming site, a recognition site for a second restriction enzyme, and a forward priming site or a part thereof including its 5′ end; or
a reverse priming site, a curvature module comprising a recognition site for the second restriction enzyme, and a forward priming site or a part thereof including its 5′ end.

In a first embodiment, the kit further comprises at least one second forward primer comprising, from its 5′ end to its 3′ end, a forward priming site or a part thereof including its 3′ end and overlapping the part of the forward priming site of the first forward primer, a barcode sequence, a recognition site for a first restriction enzyme and either at least 10 nucleotides of the 5′ end of the sequence of interest or a universal priming site.

In a second embodiment, the at least one first forward primer further comprises, at its 3′ end, either
a barcode sequence, a recognition site for a first restriction enzyme and either at least 10 nucleotides of the 5′ end of the sequence of interest or a universal priming site, and optionally, at its 5′ end, a recognition site for the third restriction enzyme and/or a recognition site for the fourth restriction enzyme; or
a recognition site for the third restriction enzyme and/or a recognition site for the fourth restriction enzyme, and either at least 10 nucleotides of the 5′ end of the sequence of interest or a universal priming site.

The present invention alternatively relates to a kit comprising at least one first reverse primer comprising, from its 5′ end to its 3′ end,
a forward priming site, a recognition site for a second restriction enzyme, and a reverse priming site or a part thereof including its 3′ end; or
a forward priming site, a curvature module comprising a recognition site for the second restriction enzyme, and a reverse priming site or a part thereof including its 3′ end.

In a first embodiment, the kit further comprises at least one second reverse primer comprising, from its 5′ end to its 3′ end, a reverse priming site or a part thereof including its 5′ end and overlapping the part of the reverse priming site of the first reverse primer, a barcode sequence, a recognition site for a first restriction enzyme and either at least 10 nucleotides of the 3′ end of the sequence of interest or a universal priming site.

In a second embodiment, the at least one first reverse primer further comprises, at its 3′ end, either
a barcode sequence, a recognition site for a first restriction enzyme and either at least 10 nucleotides of the 3′ end of the sequence of interest or a universal priming site and optionally, at its 5′ end, a recognition site for the third restriction enzyme and/or a recognition site for the fourth restriction enzyme; or
a recognition site for the third restriction enzyme and/or a recognition site for the fourth restriction enzyme, and either at least 10 nucleotides of the 3′ end of the sequence of interest or a universal priming site.

Preferably, the first forward primer or first reverse primer of the kit comprises a curvature module comprising a recognition site for the second restriction enzyme and attached to a first member of an affinity binding pair, preferably biotin. Preferably, the curvature module comprises or consists of the sequence selected from the group consisting of sequences of SEQ ID No. 1 and SEQ ID No. 2. Optionally, the kit further comprises beads or solid supports bearing the second members of the affinity binding pair, preferably avidin or streptavidins.

### BRIEF DESCRIPTION OF THE DRAWINGS

FIG. 1 depicts an embodiment of a method for preparing a nucleic acid library.

FR: Forward Primer, RP: Reverse Primer, RS1: Restriction Site 1, RS2: Restriction Site 2, BC: Barcode, SEQ: Nucleic acid sequence.

FIG. 2 depicts an embodiment of a method for preparing a nucleic acid library the use of the universal priming site.

UPS: universal sequencing primers, CM: curvature module, FR: Forward Primer, RP: Reverse Primer, RS1: Restriction Site 1, RS2: Restriction Site 2, BC: Barcode, SEQ: Nucleic acid sequence.

FIG. 3 depicts an embodiment of a method for preparing a nucleic acid library including the use of the curvature module.

UPS: universal priming site, CM: curvature module, FR: Forward Primer, RP: Reverse Primer, RS1: Restriction Site 1, RS2: Restriction Site 2, RS3: Restriction Site 3, RS4: Restriction Site 4, BC: Barcode, SEQ: Nucleic acid sequence.

### DETAILED DESCRIPTION OF THE INVENTION

The inventor has developed a new method for preparing a nucleic acid library and in particular a PET library, to be sequenced with high throughput sequencing technologies. This method is useful to prepare simultaneously a multitude of nucleic acid libraries to be sequenced, each of these libraries being characterized by a specific barcode sequence. With the method of the invention, several nucleic acid libraries may be simultaneously barcoded and sequenced. In other words, instead of preparing in parallel several libraries to be barcoded, the method of the invention allows the simultaneous preparation of several barcoded libraries by performing the step of preparing one library. The inventor provides means for introducing the barcode at the beginning of the library preparation method. The cost of sequencing is thus greatly reduced.

However, in addition to the lowering of cost and time, the sequencing library disclosed herein further presents other technical advantages. One of the greatest advantages is that the sequencing libraries prepared by the disclosed method provide information about structural arrangements of the sequences. Indeed, if a barcode is used for one particular sequence of interest, it is possible to deduce that two sequences (e.g., indicative of a gene, an exon, or the like) are born by the same amplified nucleic acid and therefore are closed. Accordingly, the method as disclosed allows the detection of translocation, deletion, reversal, duplication or insertion.

The method disclosed herein is suitable for the preparation of libraries allowing the simultaneous sequencing of a quasi-infinite number of sequences from regions of interest derived from different samples (e.g., a set of genes from different organisms, a same nucleic acid but originated from different individuals or different nucleic acids derived from a single individual).

The method allows the control of the size and orientation of the nucleic acids to be sequenced.

The method allows the elimination or decrease of chimeric product and of false positives resulting from non-specific ligations. Indeed, the method is based on amplification and intramolecular ligation or circularization steps, thereby avoiding the ligation steps used in the methods of the prior art and responsible for the inconvenience of generating chimeric products.

Libraries provided with the method disclosed herein may be used on any NGS plateform, such as the 454 Genome Sequencer (Roche Applied Science), the Illumina Genome Analyzer (Illumina, Inc.) or the SOLiD™ platform.

### DEFINITIONS

As used herein, the term “nucleic acid molecule” refers to single-stranded and double-stranded polymers of nucleotide monomers, including DNA and RNA, linked by phosphodiester bonds. The nucleic acid molecule may be linear or circular. Nucleic acid molecules may be DNA molecules, RNA molecules or DNA-RNA chimeric molecules. They can also comprise nucleobase and sugar analogs. Preferably, the term “nucleic acid molecule” is used herein to refer to a linear or circular double-stranded DNA molecule.

As used herein, the term “nucleic acid library” refers to a plurality of different single-stranded or double-stranded nucleic acid molecules, in particular DNA molecules. These molecules may be linear or circular. The nucleic acid molecules of the library may be non covalently attached to a solid support such as beads and more particularly streptavidin-coated beads or support.

The terms “upstream” and “downstream”, as used herein, refer to a position of a discrete element on a nucleic acid molecule in relation to another discrete element. A first element is upstream to a second element when located in the 5′ direction of the coding strand from said second element. A first element is downstream to a second element when located in the 3′ direction of the coding strand from said second element.

The terms “5′ end” and “3′ end”, as used herein, refers to the 3′ end or the 5′ end of the coding strand.

As used herein, the term “adjacent” refers to a position of a discrete element on a nucleic acid molecule in relation to another discrete element. A first element is adjacent to a second element when located at the 5′ end or the 3′ end of said second element. This term indicates that no other element is present between the first and the second element. In particular, the term “adjacent” means that the first and second elements are consecutive (i.e., there is no intercalating nucleotide) or are separated by a non-significant number of nucleotides, preferably by less than 20, 15, 10, 5 or 2 nucleotides.

As used herein, the term “primer” refers to a polynucleotide of about 10-200 nucleotides in length. The primer hybridizes with the target (or template) and provides a point of initiation for template-directed synthesis of a polynucleotide complementary to the target catalyzed by a polymerase enzyme such as a DNA polymerase (polymerase chain reaction amplification). PCR reactions are typically performed with a pair of primers: a forward primer (or upstream primer) and a reverse primer (or downstream primer) which delimit the region to be amplified.

The term “restriction enzyme” or “restriction endonuclease” is intended to refer to an enzyme that recognizes a specific recognition site (or restriction site) on a single-stranded or double-stranded nucleic acid molecule and cuts this molecule at a cleavage site. As used herein, the term “recognition site” (RS) refers to a specific sequence of nucleotides recognized by a restriction enzyme. As used herein, the term “cleavage site” (CS) refers to the site wherein the restriction enzyme cuts the nucleic acid molecule. Restriction enzymes may recognize and cleave nucleic acid molecule at the same site. Restriction enzymes may also cleave nucleic acid molecule at a site distant from the recognition site. According to the restriction enzyme, the cleavage site may be located downstream or upstream to the recognition site.

As used herein, the term “intra-molecular ligation” refers to the ligation of the two ends of a linear nucleic acid molecule.

As used in this specification, the term “about” refers to a range of values ±10% of the specified value. For example, “about 20” includes ±10% of 20, and refers to from 18 to 22. Preferably, the term “about” refers to a range of values ±5% of the specified value.

As used herein, the term “affinity binding pair” refers to a binding system based on two members capable of associating with each other, covalently or not, preferably non-covalently. For example, the first member may be biotin and the second member may be streptavidin or avidin. Another example of a binding pair is digoxygenin and an anti-digoxygenin antibody. Other affinity binding pairs are known in the art and contemplated herein.

As used herein, the term “circularized form of a linear nucleic acid molecule” refers to the product obtained by intra-molecular ligation of said molecule, i.e. the ligation of the 5′ end and the 3′ end of said molecule. However, this term is not associated to the real preparation of such a circularized form but is convenient for the description of the linear nucleic acid molecule.

In a first aspect, the present invention concerns a method for preparing a nucleic acid library, in particular a nucleic acid library to be sequenced. The library obtained by this method may be a DNA or a RNA library, preferably a DNA library.

The method of the invention comprises (i) the step of providing a set or a plurality of sets of linear nucleic acid molecules comprising a sequence of interest to be entirely or partially sequenced and a barcode sequence specific of each set of nucleic acid molecules and (ii) several steps of circularizing, digesting and cleaving to provide a library in which each set of nucleic acid molecules is associated with a specific barcode. Each sequencing feature provided by this library comprises a fragment of the sequence of interest and a barcode which will be sequenced simultaneously with said fragment. In a preferred embodiment, the method comprises the step i) of providing a plurality of sets of linear nucleic acid molecules comprising a sequence of interest to be entirely or partially sequenced and a barcode sequence specific of each set of nucleic acid molecules. In particular, the method comprises the step i) of providing “n” sets of linear nucleic acid molecules comprising a sequence of interest to be entirely or partially sequenced and a barcode sequence (specific of each set of nucleic acid molecules), “n” being an integer between 1 and 1000. Accordingly, “n” different sequences of interest can be simultaneously sequenced, each associated with one distinct barcode sequence. “n” is limited by the capacity of the sequencers.

The first step of the method consists of (a) providing a set or a plurality of sets of linear nucleic acid molecules, each nucleic acid molecule comprising in its circularized form and in the 5′ to 3′ direction
a sequence of interest to be entirely or partially sequenced
a reverse priming site,
a forward priming site,
a barcode sequence specific of each set of nucleic acid molecules, which is located between the forward priming site and the sequence of interest or between the sequence of interest and the reverse priming site, and
two recognition sites for two different restriction enzymes:
   a first recognition site for a first restriction enzyme which is located between the barcode sequence and the sequence of interest, and
   a second recognition site for a second restriction enzyme which is located between the reverse priming site and the forward priming site. Linear nucleic acid molecules provided in step a) may comprise from 5′ end to 3′ end:

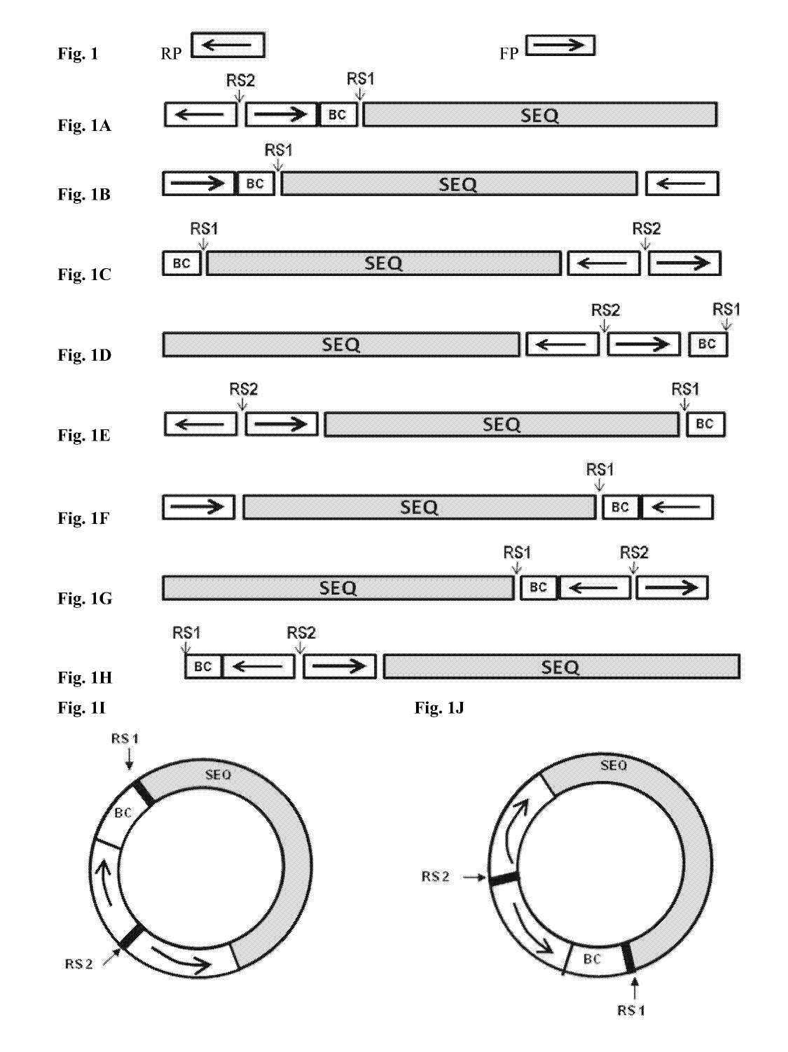

a reverse priming site, a forward priming site, a barcode sequence and a sequence of interest (FIG. 1a); or

a forward priming site, a barcode sequence, a sequence of interest and a reverse priming site (FIG. 1b); or

a barcode sequence, a sequence of interest, a reverse priming site and a forward priming site (FIG. 1c); or

a sequence of interest, a reverse priming site, a forward priming site and a barcode sequence (FIG. 1d); or

a reverse priming site, a forward priming site, a sequence of interest and a barcode sequence (FIG. 1e); or

a forward priming site, a sequence of interest, a barcode sequence and a reverse priming site (FIG. 1f); or

a sequence of interest, a barcode sequence, a reverse priming site and a forward priming site (FIG. 1g); or

a barcode sequence, a reverse priming site, a forward priming site and a sequence of interest (FIG. 1h).

When considering the nucleic acid molecule comprising in its circularized form, all the above mentioned linear forms may be recapitulated by two circularized forms: FIG. 1i is the circularized form recapitulating the linear nucleic acids of FIG. 1a-d and FIG. 1j is the circularized form recapitulating the linear nucleic acids of FIG. 1e-h.

Preferably, all nucleic acid molecules have the same conformation or arrangement of the different elements, i.e. one of the structures presented above. In a preferred embodiment, linear nucleic acid molecules provided in step a) are double stranded DNA molecules.

The reverse priming site (RP) and forward priming site (FP) are known and predetermined sequences. These priming sites are used to amplify sequencing features by using primers specific of these sites. Primers used for the amplification reaction may be universal sequencing primers. In a preferred embodiment, the sequencing reverse primer hybridizes to the reverse priming site and the sequencing forward primer hybridizes to the forward priming site. These priming sites may be easily designed by the skilled person according to the sequencing technology and with the universal primers which are intended to be used. The reverse priming site (RP) and forward priming site (FP) are called

P1 (5′CCACTACGCCTCCGCTTTCCTCTCTATGGGCAGTCGGTGAT3′) and

P2 (5′CTGCCCCGGGTTCCTCATTCTCT3′) in SOLiD system.

The sequence of interest may be any nucleic acid sequence from about 25 base pairs (bp) to 10 kbp length. In particular, the main limitation for the length of the sequence of interest is linked to the amplification limitation.

The sequence of interest can be a gene of interest or a segment thereof, or a chromosomal region of interest. One barcode is to be associated with one sequence of interest. Then, a library can be prepared from the association of one barcode to one sequence of interest by the method disclosed herein, thereby providing a library of fragments from the sequence of interest associated to the same barcode. Of course, as explained in the introduction of the detailed description, the advantage of the present method is to simultaneously prepare several libraries, wherein each initial sequence of interest is associated to one particular (and different) barcode. For instance, if the method is applied to the sequence of one particular gene of interest in several individuals or organisms (e.g., a oncogene such as p53 gene), a barcode can be attributed to each particular individual or organism. Accordingly, the gene may be simultaneously sequenced for several individuals or organisms. Alternatively, if several genes or chromosomal regions have to be sequenced for one individual, a barcode can be attributed to each particular gene or chromosomal regions.

The barcode sequence is a nucleic acid sequence comprising from 5 to 15 bp, preferably from 5 to 10 bp. This barcode sequence is specific for each set of nucleic acid molecules. Preferably, this barcode sequence does not comprise any restriction site. When considering the circularized form of the herein described nucleic acid molecules, the barcode is always adjacent to the sequence of interest, either upstream to the sequence of interest, more particularly between the forward priming site and the sequence of interest (FIG. 1i); or downstream to the sequence of interest, more particularly between the sequence of interest and the reverse priming site (FIG. 1j). When considering the different possible linear form provided in step a), that means that the barcode sequence may be located between the forward priming site and the sequence of interest (FIG. 1a and FIG. 1b), between the sequence of interest and the reverse priming site (FIG. 1f and FIG. 1g), upstream to the sequence of interest (FIG. 1c), downstream to the sequence of interest (FIG. 1e), downstream of the forward priming site (FIG. 1d) or upstream to the reverse priming site (FIG. 1h).

The nucleic acid molecules provided in step a) may further comprise a universal priming site (UPS). This universal priming site comprises or consists of a sequence of at least 10 base pairs, preferably at least 15 bp, more preferably at least 20 bp. Preferably, this universal priming site consists of 10 to 25 bp, more preferably of 15 to 20 pb. Said sequence has less than 90% identity with the genome providing the sequence of interest. Preferably, the sequence has less than 80% identity with the genome providing the sequence of interest, more preferably less than 70% identity and even more preferably, less than 60% identity. In a particular embodiment, the universal priming site comprises or consists of a sequence of at least 15 bp which has less than 80% identity with the genome providing the sequence of interest. The universal priming site may be located upstream or downstream to the sequence of interest. Linear nucleic acid molecules provided in step a) and further comprising a universal priming site (UPS) may comprise, for example, from 5′ end to 3′ end:

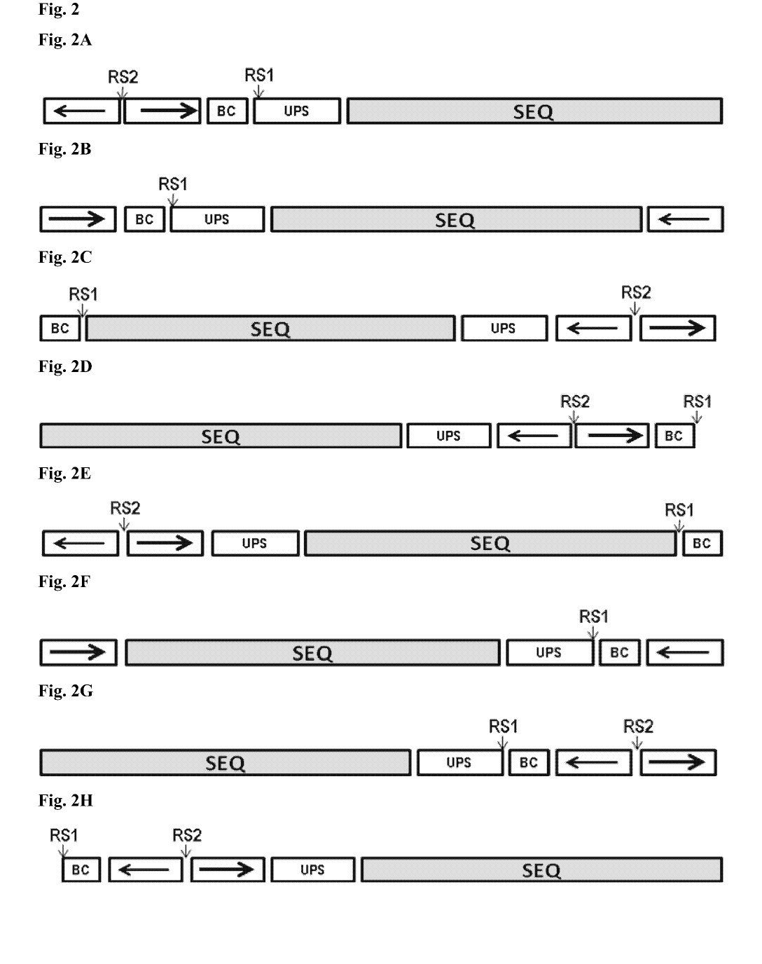

a reverse priming site, a forward priming site, a barcode sequence, a UPS sequence and a sequence of interest (FIG. 2a); or

a forward priming site, a barcode sequence, a UPS sequence, a sequence of interest and a reverse priming site (FIG. 2b); or

a barcode sequence, a sequence of interest, a UPS sequence, a reverse priming site and a forward priming site (FIG. 2c); or

a sequence of interest, a UPS sequence, a reverse priming site, a forward priming site and a barcode sequence (FIG. 2d); or

a reverse priming site, a forward priming site, a UPS sequence, a sequence of interest and a barcode sequence (FIG. 2e); or

a forward priming site, a sequence of interest, a UPS sequence, a barcode sequence and a reverse priming site (FIG. 2f); or

a sequence of interest, a UPS sequence, a barcode sequence, a reverse priming site and a forward priming site (FIG. 2g); or

a barcode sequence, a reverse priming site, a forward priming site, a UPS sequence, and a sequence of interest (FIG. 2h).

In a particular embodiment, the linear nucleic acid molecules provided in step a) comprise from 5′ end to 3′ end, a barcode sequence, a sequence of interest, a UPS, a reverse priming site and a forward priming site (FIG. 2c).

In another particular embodiment, the linear nucleic acid molecules provided in step a) comprise from 5′ end to 3′ end, a reverse priming site, a forward priming site, a UPS sequence, a sequence of interest and a barcode sequence (FIG. 2e).

In a preferred embodiment, the linear nucleic acid molecules provided in step a) comprise from 5′ end to 3′ end, a reverse priming site, a forward priming site, a barcode sequence, a UPS sequence and a sequence of interest (FIG. 2a).

In another preferred embodiment, the linear nucleic acid molecules provided in step a) comprise from 5′ end to 3′ end, a sequence of interest, a UPS sequence, a barcode sequence, a reverse priming site and a forward priming site (FIG. 2g).

The above mentioned molecules including a UPS sequence are the preferred ones. Other molecules including a UPS sequence may be contemplated, but with a less advantaging arrangement.

The nucleic acid molecules provided in step a) may further comprise a curvature module (CM) located, in their circularized form, between the reverse priming site and the forward priming site. When considering the linear nucleic acid molecules provided in step a), the curvature module located between the reverse priming site and the forward priming site or, when the reverse and forward priming sites are located at each end of the molecule (FIG. 1 or 2, b and f), is located either downstream to the reverse priming site or upstream to the forward priming site. The curvature module is a nucleotide sequence inducing a bend in the helix structure of a nucleic acid molecule. This module may be used to facilitate the circularization of nucleic acid molecules, in particular nucleic acid molecules comprising less than 250 bp. This module may comprise or consist of a nucleotide sequence obtained or derived from kinetoplast DNA minicircles found in most Trypanosoma species and in particular from kinetoplast DNA minicircles of Crithidia fasciculata. The curvature module may comprise or consist of a sequence selected from the group consisting of the sequence of SEQ ID No. 1 et SEQ ID No. 2 and a sequence having at least 90% identity with the sequence of SEQ ID No. 1 or SEQ ID No. 2. In an embodiment, the curvature module comprises or consists of a sequence selected from the group consisting of the sequence of SEQ ID No. 1 and the sequence of SEQ ID No. 2. In a particular embodiment, the curvature module comprises or consists of the sequence of SEQ ID No. 1. In another particular embodiment, the curvature module comprises or consists of the sequence of SEQ ID No. 2. (Birkenmeyer et al. 1985, Ulanovsky et al. 1986, Kitchin et al. 1986).

Accordingly, linear nucleic acid molecules provided in step a) and further comprising a curvature module may comprise, for example, from 5′ end to 3′ end:

a reverse priming site, a curvature module, a forward priming site, a barcode sequence and a sequence of interest (derived from FIG. 1a arrangement); or

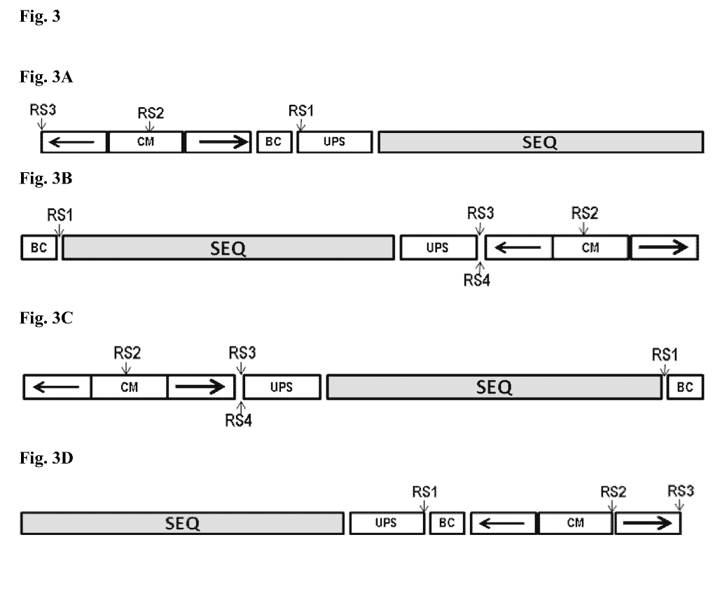

a reverse priming site, a curvature module, a forward priming site, a barcode sequence, a UPS sequence and a sequence of interest (derived from FIG. 2a arrangement; FIG. 3a); or

a curvature module, a forward priming site, a barcode sequence, a sequence of interest and a reverse priming site (derived from FIG. 1b arrangement); or

a forward priming site, a barcode sequence, a sequence of interest, a reverse priming site and a curvature module (derived from FIG. 1b arrangement); or

a curvature module, a forward priming site, a barcode sequence, a UPS sequence, a sequence of interest and a reverse priming site (derived from FIG. 2b arrangement); or

a forward priming site, a barcode sequence, a UPS sequence, a sequence of interest, a reverse priming site and a curvature module (derived from FIG. 2b arrangement); or

a forward priming site, a barcode sequence, a sequence of interest, a UPS sequence, a reverse priming site and a curvature module; or

a barcode sequence, a sequence of interest, a reverse priming site, a curvature module and a forward priming site (derived from FIG. 1c arrangement); or

a barcode sequence, a sequence of interest, a UPS sequence, a reverse priming site, a curvature module and a forward priming site (derived from FIG. 2c arrangement; FIG. 3b); or

a sequence of interest, a reverse priming site, a curvature module, a forward priming site and a barcode sequence (derived from FIG. 1d arrangement); or

a sequence of interest, a UPS sequence, a reverse priming site, a curvature module, a forward priming site and a barcode sequence (derived from FIG. 2d arrangement); or

a reverse priming site, a curvature module, a forward priming site, a sequence of interest and a barcode sequence (derived from FIG. 1e arrangement); or

a reverse priming site, a curvature module, a forward priming site, a UPS sequence, a sequence of interest and a barcode sequence (derived from FIG. 2e arrangement; FIG. 3c); or

a curvature module, a forward priming site, a sequence of interest, a barcode sequence and a reverse priming site (derived from FIG. 1f arrangement); or

a curvature module, a forward priming site, a sequence of interest, a UPS sequence, a barcode sequence and a reverse priming site (derived from FIG. 2f arrangement); or

a curvature module, a forward priming site, a UPS sequence, a sequence of interest, a barcode sequence and a reverse priming site; or

a forward priming site, a sequence of interest, a barcode sequence, a reverse priming site and a curvature module (derived from FIG. 1f arrangement); or

a forward priming site, a sequence of interest, a UPS sequence, a barcode sequence, a reverse priming site and a curvature module (derived from FIG. 2f arrangement); or

a sequence of interest, a barcode sequence, a reverse priming site, a curvature module and a forward priming site (derived from FIG. 1g arrangement); or

a sequence of interest, a UPS sequence, a barcode sequence, a reverse priming site, a curvature module and a forward priming site (derived from FIG. 2g arrangement; FIG. 3d); or

a barcode sequence, a reverse priming site, a curvature module, a forward priming site and a sequence of interest (derived from FIG. 1h arrangement); or

a barcode sequence, a reverse priming site, a curvature module, a forward priming site, a UPS sequence and a sequence of interest (derived from FIG. 2h arrangement).

In a particular embodiment, the linear nucleic acid molecules provided in step a) comprise from 5′ end to 3′ end, a barcode sequence, a sequence of interest, a UPS sequence, a reverse priming site, a curvature module and a forward priming site (FIG. 3b).
In another particular embodiment, the linear nucleic acid molecules provided in step a) comprise from 5′ end to 3′ end, a reverse priming site, a curvature module, a forward priming site, a UPS sequence, a sequence of interest and a barcode sequence (FIG. 3c).
In a preferred embodiment, the linear nucleic acid molecules provided in step a) comprise from 5′ end to 3′ end, a reverse priming site, a curvature module, a forward priming site, a barcode sequence, a UPS sequence and a sequence of interest (FIG. 3a).
In another preferred embodiment, the linear nucleic acid molecules provided in step a) comprise from 5′ end to 3′ end, a sequence of interest, a UPS sequence, a barcode sequence, a reverse priming site, a curvature module and a forward priming site (FIG. 3d).

The nucleic acid molecules provided in step a) comprise a recognition site for a first restriction enzyme.

The recognition site for the first restriction enzyme is located, when considering the circularized form of the nucleic acid molecules, between the barcode and the sequence of interest.

In an embodiment, the linear nucleic acid molecules provided in step a) comprise a barcode sequence adjacent to the sequence of interest (FIG. 1a-c, e-g; FIGS. 2c and 2e, FIGS. 3b and c), and the recognition site for the first restriction enzyme is located between the barcode sequence and the sequence of interest.

In another embodiment, the linear nucleic acid molecules provided in step a) comprise a barcode sequence in 5′ end and a sequence of interest in 3′ end (FIG. 1h and FIG. 2h), and the recognition site for the first restriction enzyme is located upstream to the barcode sequence or downstream to the sequence of interest, but preferably upstream to the barcode sequence.

In an additional embodiment, the linear nucleic acid molecules provided in step a) comprise a barcode sequence in 3′ end and a sequence of interest in 5′ end (FIG. 1d and FIG. 2d) and the recognition site for the first restriction enzyme is located downstream to the barcode sequence or upstream to the sequence of interest, but preferably downstream to the barcode sequence.

In a further embodiment, the linear nucleic acid molecules provided in step a) comprise a universal priming site (UPS) between the barcode sequence and the sequence of interest (FIGS. 2a, 2b, 2f and 2g, FIGS. 3a and 3d) and the recognition site for the first restriction enzyme is located between the universal priming site (UPS) and the barcode sequence or into said universal priming site (preferably near the barcode sequence).

The nucleic acid molecules provided in step a) further comprise a recognition site for a second restriction enzyme which is located, when considering the circularized form of the nucleic acid molecules, between the two priming sites (e.g., FIG. 1i and 1j). In a particular embodiment, the nucleic acid molecules further comprise a curvature module located between the reverse and forward priming sites and the second recognition site for the second restriction enzyme is located into said curvature module (FIG. 3a-d).

In a very particular embodiment, the nucleic acid molecules further comprise a curvature module located between the reverse and forward priming sites, the second recognition site for the second restriction enzyme is located into said curvature module and the second restriction enzyme is PacI.

The nucleic acid molecules provided in step a) may further comprise a recognition site for a third restriction enzyme. The third restriction enzyme is a non palindromic endonuclease which cleaves DNA at a defined distance from its recognition site.

In an embodiment, nucleic acid molecules provided in step a) comprise, when considered in their circularized form and in the 5′ to 3′ direction, a sequence of interest, a reverse priming site, a forward priming site and a barcode sequence located between the forward priming site and the sequence of interest (FIG. 1i), and the recognition site for the third restriction enzyme is located upstream to the reverse priming site. In this embodiment, the third restriction enzyme cuts nucleic acid molecules in a cleavage site upstream to its recognition site, i.e. in the sequence of interest in the circularized form of molecules.

In another embodiment, nucleic acid molecules provided in step a) comprise in their circularized form and in the 5′ to 3′ direction, a reverse priming site, a forward priming site, a sequence of interest and a barcode sequence located between the sequence of interest and the reverse priming site (FIG. 1j), and the recognition site for the third restriction enzyme is located downstream to the forward priming site. In this embodiment, the third restriction enzyme cuts DNA (i.e., a double-strand cut) in a cleavage site downstream to its recognition site, i.e. in the sequence of interest in the circularized form of molecules.

In a particular embodiment, the third restriction enzyme is selected from the group consisting of EcoP15I, MmeI, NmeAIII, AcuI, BbvI, BceAI, BpmI, BpuEI, BseRI, BsgI, BsmFI, BtgZI, EciI, FokI, HgaI, I-CeuI, I-SceI, PI-PspI and PI-SceI. In a more particular embodiment, the third restriction enzyme is selected from the group consisting EcoP15I, MmeI and NmeAIII. In a preferred embodiment, the third restriction enzyme is EcoP15I.

The nucleic acid molecules provided in step a) may further comprise a recognition site for a fourth restriction enzyme.

In an embodiment, nucleic acid molecules provided in step a) comprise, when considered in their circularized form and in the 5′ to 3′ direction, a sequence of interest, a reverse priming site, a forward priming site and a barcode sequence located between the forward priming site and the sequence of interest (FIG. 1i), and the recognition site for the fourth restriction enzyme is located upstream to the reverse priming site.

In another embodiment, nucleic acid molecules provided in step a) comprise, when considered in their circularized form and in the 5′ to 3′ direction, a reverse priming site, a forward priming site, a sequence of interest and a barcode sequence located between the sequence of interest and the reverse priming site (FIG. 1j), and the recognition site for the fourth restriction enzyme is located downstream to the forward priming site.

The first, second, third and fourth restriction enzymes may be chosen in order to have infrequent/occasional or no additional cleavage site in the sequence of interest and in any other part of the nucleic acid molecules. Preferably, restriction enzymes are chosen in order to have only one cleavage site in the nucleic acid molecules.

Preferably, the first, second and fourth restriction enzymes cut DNA (i.e., a double-strand cut) in their recognition sites or at a distance from these sites of less than 5 nucleotides. More preferably, the first, second and fourth restriction enzymes cut DNA in their recognition sites.

These restriction enzymes may be chosen in order to have no cleavage site or a low frequency of cutting in the sequence of interest. The site frequency of a restriction enzyme in sequenced genomes may be easily found by the skilled person on available databases (such as REBASE http://rebase.neb.com). Restriction enzymes may thus be chosen in order to have a low frequency of cutting in the genome providing the sequence of interest.

In a particular embodiment, the first restriction enzyme is selected from the group consisting of SrfI, SbfI, AscI, NotI, BssHII, SacII, FseI, SmaI. In a preferred embodiment, the first restriction enzyme is selected from the group consisting of SrfI and SbfI.

In a particular embodiment, the second restriction enzyme is selected from the group consisting of PacI, AscI, NotI, BssHII, SacII, FseI, SmaI. In a preferred embodiment, the second restriction enzyme is PacI.

In a particular embodiment, the fourth restriction enzyme is selected from the group consisting of PmeI, AscI, NotI, BssHII, SacII, FseI, SmaI. In a preferred embodiment, the fourth restriction enzyme is selected from the group consisting of PmeI and AscI.

The nucleic acid molecules provided in step a) may further comprise a binding site for a first member of an affinity binding pair. Preferably, this binding site is located in the region from the 5′ end of the reverse priming site to the 3′ end of the forward priming site. In an embodiment, the nucleic acid molecules provided in step a) further comprise a curvature module located between the reverse and forward priming sites and said curvature module comprises a binding site for a first member of an affinity binding pair.

The nucleic acid molecules provided in step a) may also be attached to a first member of an affinity binding pair. Preferably, the first member of an affinity binding pair is attached in the region from the 5′ end of the reverse priming site to the 3′ end of the forward priming site. In an embodiment, the nucleic acid molecules provided in step a) comprise a curvature module located between the reverse and forward priming sites and the first member of an affinity binding pair is attached to said curvature module.

The affinity binding pair may be, for example, digoxigenin—anti digoxigenin antibody or biotin—avidin/streptavidin. Preferably, the first member of the affinity binding pair is biotin and the second member is streptavidin. In a particular embodiment, the nucleic acid molecules provided in step a) comprise a curvature module comprising a biotin-modified thymidine. As a specific example, the biotin-modified thymidine may be thymidine 26 of SEQ ID No. 1 or thymidine 13 of SEQ ID No. 2.

The nucleic acid molecules provided in step a) of the method of the invention may be obtained by one or several amplification reactions Amplification may be performed by any technique, including, but not limited to, PCR, RT-PCR, Qβ-replicase amplification (Cahill et al., 1991; Chetverin and Spirin, 1995; Katanaev et al., 1995), the ligase chain reaction (LCR) (Landegren et al., 1988; Barany, 1991), the self-sustained sequence replication system (Fahy et al., 1991), strand displacement amplification (Walker et al., 1992), nucleic acid sequence-based amplification (NASBA) (Compton, 1991), loop-mediated isothermal amplification (Notomi et al., 2000), rolling circle amplification (RCA) (Blanco et al., 1989) and hyperbranched rolling circle amplification (HRCA) (Lizardi et al., 1998). Preferably amplification is by PCR or RT-PCR. Preferably, a high-fidelity polymerase is used and the error rate of the polymerase is less than 10⁻⁵, more preferably less than 10⁻⁶, and even more preferably less than 5·10⁻⁷. Preferably, Platinum Taq DNA Polymerase High Fidelity (Invitrogen), Phusion Hot Start High-Fidelity DNA Polymerase (New England BioLabs), FastStart High Fidelity PCR System (Roche).

The template for this amplification may be a DNA or RNA molecule. For the PCR or RT-PCR amplification, a set of primers is needed. A set of primers comprise at least two primers: a forward primer and a reverse primer. The set of primers may be easily designed by the skilled person according to the structure of the linear nucleic acid molecules to be obtained, the sequence of interest and the number of amplification reactions.

The nucleic acid molecules provided in step a) may be obtained by one amplification reaction, preferably by one RT-PCR or PCR reaction. The forward primer for this reaction comprises the region of the linear nucleic acid molecules provided in step a) which is upstream to the sequence of interest and at least 10, preferably 12, 15, 20 or 25, nucleotides of the 5′ end of the sequence of interest. The reverse primer comprises the region of the nucleic acid molecules which is downstream to the sequence of interest and at least 10, preferably 12, 15, 20 or 25, nucleotides of the 3′ end of the sequence of interest.

In a particular embodiment with nucleic acid molecules with an arrangement as shown FIG. 1a, optionally with a curvature module, the nucleic acid molecules provided in step a) are obtained by one amplification reaction with a set of primers comprising a reverse primer specific of the 3′ end of the sequence of interest and a forward primer comprising from its 5′ end to its 3′ end:
a reverse priming site, a recognition site for the second restriction enzyme, a forward priming site, a barcode sequence, a recognition site for the first restriction enzyme and at least 10 nucleotides of the 5′ end of the sequence of interest; or
a reverse priming site, a curvature module comprising a recognition site for the second restriction enzyme, a forward priming site, a barcode sequence, a recognition site for the first restriction enzyme and at least 10 nucleotides of the 5′ end of the sequence of interest. Optionally, the forward primer may comprise at its 5′ end a recognition site for the third restriction enzyme and/or a recognition site for the fourth restriction enzyme.

In a more particular embodiment, the forward primer is attached to a first member of an affinity binding pair. Preferably, the forward primer is attached to biotin. More preferably, the forward primer comprises a curvature module attached to biotin.

In a preferred embodiment, the nucleic acid molecules provided in step a) are obtained by one amplification reaction with a set of primers comprising a reverse primer specific of the 3′ end of the sequence of interest and a forward primer comprising from its 5′ end to its 3′ end, a reverse priming site, a curvature module attached to biotin and comprising a recognition site for the second restriction enzyme, a forward priming site, a barcode sequence, a recognition site for the first restriction enzyme and at least 10 nucleotides of the 5′ end of the sequence of interest. Preferably, the forward primer further comprises at its 5′ end a recognition site for the third restriction enzyme and, optionally a recognition site for the fourth restriction enzyme.

In another particular embodiment with nucleic acid molecules with an arrangement as shown FIG. 1g, optionally with a curvature module, the nucleic acid molecules provided in step a) are obtained by one amplification reaction with a set of primers comprising a forward primer specific to the 5′ end of the sequence of interest and a reverse primer comprising, from its 5′ end to its 3′ end:
a forward priming site, a recognition site for the second restriction enzyme, a reverse priming site, a barcode sequence, a recognition site for the first restriction enzyme and at least 10 nucleotides of the 3′ end of the sequence of interest; or
a forward priming site, a curvature module comprising a recognition site for the second restriction enzyme, a reverse priming site, a barcode sequence, a recognition site for the first restriction enzyme and at least 10 nucleotides of the 3′ end of the sequence of interest. Optionally, the reverse primer may comprise at its 5′ end a recognition site for the third restriction enzyme and/or a recognition site for the fourth restriction enzyme.

In a more particular embodiment, the reverse primer is attached to a first member of an affinity binding pair. Preferably, the reverse primer is attached to biotin. More preferably, the reverse primer comprises a curvature module attached to biotin.

In a preferred embodiment, the nucleic acid molecules provided in step a) are obtained by one amplification reaction with a set of primers comprising a forward primer specific of the 5′ end of the sequence of interest and a reverse primer comprising from its 5′ end to its 3′ end a forward priming site, a curvature module attached to biotin and comprising a recognition site for the second restriction enzyme, a reverse priming site, a barcode sequence, a recognition site for the first restriction enzyme and at least 10 nucleotides of the 3′ end of the sequence of interest. Preferably, the reverse primer comprises at its 5′ end a recognition site for the third restriction enzyme and, optionally a recognition site for the fourth restriction enzyme

In another particular embodiment with nucleic acid molecules with an arrangement as shown FIG. 1c, optionally with a curvature module, the nucleic acid molecules provided in step a) are obtained by one amplification reaction with a set of primers comprising a forward primer comprising, from its 5′end to its 3′ end, a barcode sequence, a recognition site for the first restriction enzyme and at least 10 nucleotides of the 5′ end of the sequence of interest, and a reverse primer comprising, from its 5′ end to its 3′ end:
a forward priming site, a recognition site for the second restriction enzyme, a reverse priming site and at least 10 nucleotides of the 3′ end of the sequence of interest; or
a forward priming site, a curvature module optionally attached to biotin and comprising a recognition site for the second restriction enzyme, a reverse priming site and at least 10 nucleotides of the 3′ end of the sequence of interest. Optionally, the forward primer may comprise a recognition site for the third restriction enzyme and/or a recognition site for the fourth restriction enzyme, between the reverse priming site and the 3′ end of the sequence of interest.

In another particular embodiment with nucleic acid molecules with an arrangement as shown FIG. 1e, optionally with a curvature module, the nucleic acid molecules provided in step a) are obtained by one amplification reaction with a set of primers comprising a reverse primer comprising, from its 5′ end to its 3′ end, a barcode sequence, a recognition site for the first restriction enzyme and at least 10 nucleotides of the 3′ end of the sequence of interest, and a forward primer comprising, from its 5′ end to its 3′ end:
a reverse priming site, a recognition site for the second restriction enzyme, a forward priming site and at least 10 nucleotides of the 5′ end of the sequence of interest; or
a reverse priming site, a curvature module optionally attached to biotin and comprising a recognition site for the second restriction enzyme, a forward priming site and at least 10 nucleotides of the 5′ end of the sequence of interest. Optionally, the forward primer may comprise a recognition site for the third restriction enzyme and/or a recognition site for the fourth restriction enzyme, between the forward priming site and the 5′ end of the sequence of interest. Preferably, the forward primer comprises a recognition site for the third restriction enzyme and a recognition site for the fourth restriction enzyme.

The one skilled in the art may easily design an appropriate set of primers for preparing the nucleic acid molecules of step a) by one amplification reaction based on the same rules. The different sets of primers may be prepared by changing the barcode sequence for each different sequence of interest and, of course, by adapting the sequence specific of the targeted sequence of interest.

The nucleic acid molecules provided in step a) may also be obtained by several amplification reactions. These amplification reactions may be performed successively or simultaneously in the same reaction mix. If the targets to be amplified are very large, these amplification are performed simultaneously by using RainStorm platform, developed by RainDance Technologies (Mamanova et al., 2010), Primers to be used in these reactions may be easily designed by the skilled person. Indeed, each set of primers is to contain at least one primer sequence overlapping with another one (overlapping forward primers and/or overlapping reverse primers, preferably not both simultaneously). By overlapping primers or overlap is intended herein that the overlap is sufficient to prime the amplification. Accordingly, the overlap is of at least 10, 15 or 20 nucleotides.

In a preferred embodiment of the method, the nucleic acid molecules provided in step a) have, either at their 5′ end or at their 3′ end, a group of elements including, from 5′ end to 3′ end, a reverse priming site, a recognition site for the second restriction enzyme, a forward priming site. More preferably, they have a group of elements including, from 5′ end to 3′ end, a reverse priming site, a curvature module optionally attached to a first member of an affinity binding pair, for example biotin, and comprising a recognition site for the second restriction enzyme, a forward priming site. Preferably, the amplification reactions may use at least two different sets of primers with either the forward primers overlapping in the forward priming site or the reverse primers being overlapping in the reverse priming site, depending on the location of this group of elements. In particular, when this group of elements is at the 5′ end of the nucleic acid molecule provided at step a), the sets of primers include forward primers overlapping in the forward priming site. Alternatively, when this group of elements is at the 3′ end of the nucleic acid molecule provided at step a), the sets of primers include reverse primers being overlapping in the reverse priming site.

In a particular embodiment, the nucleic acid molecules provided in step a) are obtained by amplification reactions with a set of primers comprising a reverse primer specific of the 3′ end of the sequence of interest and
a first forward primer comprising, from its 5′ end to its 3′ end, the forward priming site or a first part thereof including its 3′ end, a barcode sequence, a recognition site for the first restriction enzyme and at least 10 nucleotides of the 5′ end of the sequence of interest; and
a second forward primer comprising:
   from its 5′ end to its 3′ end, a reverse priming site, a recognition site for the second restriction enzyme and the forward priming site or a second part thereof overlapping the first part thereof and including its 5′ end, or
   from its 5′ end to its 3′ end, a reverse priming site, a curvature module comprising a recognition site for the second restriction enzyme, the curvature module being optionally attached to a first member of an affinity binding pair, the forward priming site or a second part thereof overlapping the first part thereof and including its 5′ end.

Optionally, the second forward primer may comprise at its 5′ end a recognition site for the third restriction enzyme and/or a recognition site for the fourth restriction enzyme.

In another particular embodiment, the nucleic acid molecules provided in step a) are obtained by amplification reactions with a set of primers comprising a reverse primer comprising from its 5′ end to its 3′ end, a barcode sequence, a recognition site for the first restriction enzyme and at least 10 nucleotides of the 3′ end of the sequence of interest and
a first forward primer comprising, from its 5′ end to its 3′ end, the forward priming site or a first part thereof including its 3′ end, and at least 10 nucleotides of the 5′ end of the sequence of interest; and
a second forward primer comprising:
   from its 5′ end to its 3′ end, a reverse priming site, a recognition site for the second restriction enzyme and the forward priming site or a second part thereof overlapping the first part thereof and including its 5′ end, or
   from its 5′ end to its 3′ end, a reverse priming site, a curvature module comprising a recognition site for the second restriction enzyme, the curvature module being optionally attached to a first member of an affinity binding pair, the forward priming site or a second part thereof overlapping the first part thereof and including its 5′ end.

Optionally, the second forward primer may comprise at its 5′ end a recognition site for the third restriction enzyme and/or a recognition site for the fourth restriction enzyme.

In a further particular embodiment, the nucleic acid molecules provided in step a) are obtained by amplification reactions with a set of primers comprising a forward primer comprising from its 5′ end to its 3′ end, a barcode sequence, a recognition site for the first restriction enzyme and at least 10 nucleotides of the 5′ end of the sequence of interest, and
a first reverse primer comprising, from its 5′ end to its 3′ end, the reverse priming site or a first part thereof including its 5′ end and at least 10 nucleotides of the 3′ end of the sequence of interest; and,
a second reverse primer comprising:
   from its 5′ end to its 3′ end, a forward priming site, a recognition site for the second restriction enzyme, the reverse priming site or a second part thereof overlapping the first part thereof and including its 3′ end; or
   from its 5′ end to its 3′ end, a forward priming site, a curvature module comprising a recognition site for the second restriction enzyme, the curvature module being optionally attached to a first member of an affinity binding pair, the reverse priming site or a second part thereof overlapping the first part thereof and including its 3′ end.

Optionally, the second reverse primer may comprise at its 5′ end a recognition site for the third restriction enzyme and/or a recognition site for the fourth restriction enzyme.

In an additional particular embodiment, the nucleic acid molecules provided in step a) are obtained by amplification reactions with a set of primers comprising a forward primer comprising from at least 10 nucleotides of the 5′ end of the sequence of interest, and
a first reverse primer comprising, from its 5′ end to its 3′ end, the reverse priming site or a first part thereof including its 5′ end, a barcode sequence, a recognition site for the first restriction enzyme and at least 10 nucleotides of the 3′ end of the sequence of interest; and,
a second reverse primer comprising:
   from its 5′ end to its 3′ end, a forward priming site, a recognition site for the second restriction enzyme, the reverse priming site or a second part thereof overlapping the first part thereof and including its 3′ end; or
   from its 5′ end to its 3′ end, a forward priming site, a curvature module comprising a recognition site for the second restriction enzyme, the curvature module being optionally attached to a first member of an affinity binding pair, the reverse priming site or a second part thereof overlapping the first part thereof and including its 3′ end.

Optionally, the second reverse primer may comprise at its 5′ end a recognition site for the third restriction enzyme and/or a recognition site for the fourth restriction enzyme.

In an advantageous embodiment, the method uses a universal priming site (UPS) sequence. The use of UPS allows the design of shorter primers specific to the sequences of interest, thereby lowering the cost. Indeed, preferred first primers of the method include an UPS sequence and at least 10 nucleotides of the 5′ or 3′ end of the sequence of interest. The other primers are not specific to the sequences of interest and can be used as standardized products, convenient for preparing kits suitable for performing the methods as disclosed herein.

In a particular embodiment, the nucleic acid molecules provided in step a) are obtained by amplification reactions with a set of primers comprising a reverse primer specific of the 3′ end of the sequence of interest, and:
a first forward primer comprising, from its 5′ end to its 3′ end, a universal priming site and at least 10 nucleotides of the 5′ end of the sequence of interest; and
a second forward primer comprising:
   from its 5′ end to its 3′ end, a reverse priming site, a recognition site for the second restriction enzyme, a forward priming site, a barcode sequence, a recognition site for the first restriction enzyme and the universal priming site, or
   from its 5′ end to its 3′ end, a reverse priming site, a curvature module comprising a recognition site for the second restriction enzyme, the curvature module being optionally attached to a first member of an affinity binding pair, a forward priming site, a barcode sequence, a recognition site for the first restriction enzyme and the universal priming site.

Optionally, the second forward primer may comprise at its 5′ end a recognition site for the third restriction enzyme and/or a recognition site for the fourth restriction enzyme.

In another particular embodiment, the nucleic acid molecules provided in step a) are obtained by amplification reactions with a set of primers comprising a reverse primer specific of the 3′ end of the sequence of interest, and:
a first forward primer comprising from its 5′ end to its 3′ end a universal priming site and at least 10 nucleotides of the 5′ end of the sequence of interest; and
a second forward primer comprising from its 5′ end to its 3′ end, the forward priming site or a first part thereof including its 3′ end, a barcode sequence, a recognition site for the first restriction enzyme and the universal priming site; and
a third forward primer comprising:
   from its 5′ end to its 3′ end, a reverse priming site, a recognition site for the second restriction enzyme and the forward priming site or a second part thereof overlapping the first part thereof and including its 5′ end, or
   from its 5′ end to its 3′ end, a reverse priming site, a curvature module comprising a recognition site for the second restriction enzyme, the curvature module being optionally attached to a first member of an affinity binding pair, and the forward priming site or a second part thereof overlapping the first part thereof and including its 5′ end.

Optionally, the third forward primer may comprise at its 5′ end a recognition site for the third restriction enzyme and/or a recognition site for the fourth restriction enzyme.

In another particular embodiment, the nucleic acid molecules provided in step a) are obtained by amplification reactions with a set of primers comprising a forward primer comprising from its 5′ end to its 3′ end, a barcode sequence, a recognition site for the first restriction enzyme and at least 10 nucleotides of the 5′ end of the sequence of interest, and
a first reverse primer comprising, from its 5′ end to its 3′ end, a universal priming site and at least 10 nucleotides of the 3′ end of the sequence of interest; and
a second reverse primer comprising:
   from its 5′ end to its 3′ end, a forward priming site, a recognition site for the second restriction enzyme, a reverse priming site and the universal priming site, or
   from its 5′ end to its 3′ end, a forward priming site, a curvature module comprising a recognition site for the second restriction enzyme, the curvature module being optionally attached to a first member of an affinity binding pair, a reverse priming site and the universal priming site.

Optionally, the second reverse primer may comprise between the universal priming site and the reverse priming site, a recognition site for the third restriction enzyme and/or a recognition site for the fourth restriction enzyme.

In another particular embodiment, the nucleic acid molecules provided in step a) are obtained by amplification reactions with a set of primers comprising a reverse primer comprising from its 5′ end to its 3′ end, a barcode sequence, a recognition site for the first restriction enzyme and at least 10 nucleotides of the 3′ end of the sequence of interest, and
a first forward primer comprising, from its 5′ end to its 3′ end, a universal priming site and at least 10 nucleotides of the 5′ end of the sequence of interest, and
a second forward primer comprising:
   from its 5′ end to its 3′ end, a reverse priming site, a recognition site for the second restriction enzyme, a forward priming site and the universal priming site, or
   from its 5′ end to its 3′ end, a reverse priming site, a curvature module comprising a recognition site for the second restriction enzyme, the curvature module being optionally attached to a first member of an affinity binding pair, a forward priming site and the universal priming site.

Optionally the second forward primer may comprise a recognition site for the third restriction enzyme and/or a recognition site for the fourth restriction enzyme, located between the forward priming site and the universal priming site.

In another particular embodiment, the nucleic acid molecules provided in step a) are obtained by amplification reactions with a set of primers comprising a forward primer specific of the 5′ end of the sequence of interest, and:
a first reverse primer comprising from its 5′ end to its 3′ end, a universal priming site and at least 10 nucleotides of the 3′ end of the sequence of interest, and
a second reverse primer comprising:
   from its 5′ end to its 3′ end, a forward priming site, a recognition site for the second restriction enzyme, a reverse priming site, a barcode sequence, a recognition site for the first restriction enzyme and the universal priming site, or
   from its 5′ end to its 3′ end, a forward priming site, a curvature module comprising a recognition site for the second restriction enzyme, the curvature module being optionally attached to a first member of an affinity binding pair, a reverse priming site, a barcode sequence, a recognition site for the first restriction enzyme and the universal priming site.

Optionally the second reverse primer may comprise a recognition site for the third restriction enzyme and/or a recognition site for the fourth restriction enzyme, located at its 5′ end.

In another particular embodiment, the nucleic acid molecules provided in step a) are obtained by amplification reactions with a set of primers comprising a forward primer specific of the 5′ end of the sequence of interest, and:
a first reverse primer comprising from its 5′ end to its 3′ end, a universal priming site and at least 10 nucleotides of the 3′ end of the sequence of interest, and,
a second reverse primer comprising from its 5′ end to its 3′ end, the reverse priming site or a first part thereof including its 5′ end, a barcode sequence, a recognition site for the first restriction enzyme, the universal priming site, and
a third reverse primer comprising:
   from its 5′ end to its 3′ end, a forward priming site, a recognition site for the second restriction enzyme and the reverse priming site or a second part thereof overlapping the first part thereof and including its 3′ end, or
   from its 5′ end to its 3′ end, a forward priming site, a curvature module comprising a recognition site for the second restriction enzyme, the curvature module being optionally attached to a first member of an affinity binding pair, and the reverse priming site or a second part thereof overlapping the first part thereof and including its 3′ end.

Optionally the third reverse primer may comprise a recognition site for the third restriction enzyme and/or a recognition site for the fourth restriction enzyme, located at its 5′ end.

Optionally, after amplification reaction(s), excess single-stranded primers may be degraded, for example, by using exonuclease I or any other nuclease which is specific of single-stranded DNA.

The further steps of the method may be carried out on the plurality of sets of linear nucleic acid sequences, thereby optimizing the time and money cost of preparing a library.

In step b) of the method of the invention, linear nucleic acid molecules provided in step a) or a′) are circularized by intra-molecular ligation. Accordingly, two different types of circularized molecules may be obtained, the first type comprises the entire sequence of interest and the second type comprises the sequence of interest already truncated at one of its end (i.e., when a step a′) has been performed). This ligation may be performed by any method known by the skilled person. Preferably, the intra-molecular ligation is performed by using a DNA ligase, such as T4 DNA ligase, in conditions as described in the article of Collins and Weissman, 1984. The conditions used in this step prevent any inter-molecular ligation. Inter-molecular ligation may be prevented for instance by using the appropriate dilution (e.g., limit dilution) or, when the nucleic acid molecules are attached to a first member of an affinity binding pair, by binding the nucleic acid molecules to a solid support bearing the first member of an affinity binding pair.

In step c) of the method of the invention, circularized nucleic acid molecules obtained from step b) are digested with a restriction enzyme in order to provide a linearized form thereof, preferably by the first restriction enzyme. This restriction enzyme cuts circularized nucleic acid molecules between the barcode and the sequence of interest. This digestion produces linear nucleic acid molecules comprising at one end the barcode sequence and at the other end the sequence of interest. When a universal priming site is present between the barcode sequence and the sequence of interest, the first restriction enzyme cuts between the universal priming site and the barcode sequence or into the universal priming site, preferably near the end adjacent to the barcode sequence.

When the linear nucleic acid molecules provided in step a) comprises in their circularized form and in the 5′ end to 3′ end direction, a sequence of interest, a reverse priming site, a forward priming site and a barcode sequence located between the forward priming site and the sequence of interest (FIG. 1i), digestion with the first restriction enzyme provides linear nucleic acid molecules comprising the sequence of interest at their 5′ end and the barcode sequence at their 3′ end.

When the linear nucleic acid molecules provided in step a) comprises in their circularized form and in the 5′ end to 3′ end direction, a reverse priming site, a forward priming site, a sequence of interest and a barcode sequence located between the reverse priming site and the sequence of interest (FIG. 1j), digestion with the first restriction enzyme provides linear nucleic acid molecules comprising the sequence of interest at their 3′ end and the barcode sequence at their 5′ end.

In step d) of the method of the invention, digested nucleic acid molecules obtained from step c) are cleaved in the sequence of interest. This cleavage may be performed by enzymatic or physical methods.

The cleavage may be performed by using a sequence independent technique of cleavage such as, for example, sonication, nebulization, French Press or by using the Hydroshear® system (Genomic Solutions®). Preferably, the cleavage is performed by sonication. This cleavage generates random nucleic acid fragments of specific sizes. The size of fragmented molecules may be chosen by the skilled person according to the intended use of the library prepared by the method of the invention. Cleaved molecules may be separated by electrophoresis and molecules of the desired size may be purified from the electrophoresis gel.

In one embodiment, the linear nucleic acid molecules provided in step a) comprises in their circularized form and in the 5′ end to 3′ end direction, a sequence of interest, a reverse priming site, a forward priming site and a barcode sequence located between the forward priming site and the sequence of interest (FIG. 1). In this embodiment, digestion with the first restriction enzyme in step c) provides linear nucleic acid molecules comprising at their 5′ end, the 5′ end of the sequence of interest. Cleavage of these nucleic acid molecules thus provides linear nucleic acid molecules comprising the sequence of interest truncated in 5′.

In another embodiment, the linear nucleic acid molecules provided in step a) comprises in their circularized form and in the 5′ end to 3′end direction, a reverse priming site, a forward priming site, a sequence of interest and a barcode sequence located between the reverse priming site and the sequence of interest (FIG. 2). In this embodiment, digestion with the first restriction enzyme in step c) provides linear nucleic acid molecules comprising, at their 3′ end, the 3′ end of the sequence of interest. Cleavage of these nucleic acid molecules thus provides linear nucleic acid molecules comprising the sequence of interest truncated in 3′.

The cleavage may also be performed by using restriction enzyme. In this case, the nucleic acid molecules provided in step a) comprise a recognition site for a third restriction enzyme as described above.

In a particular embodiment, the linear nucleic acid molecules provided in step a) comprises in their circularized form, a barcode sequence located between the forward priming site and the sequence of interest and a recognition site for the third restriction enzyme between the reverse priming site and the sequence of interest. In this embodiment, digestion of nucleic acid molecules obtained from step c) with the third enzyme provides nucleic acid molecules comprising a sequence of interest truncated in 5′.

In another particular embodiment, the linear nucleic acid molecules provided in step a) comprises in their circularized form, a barcode sequence located between the reverse priming site and the sequence of interest and a recognition site for the third restriction enzyme between the forward priming site and the sequence of interest. In this embodiment, digestion of nucleic acid molecules obtained from step c) with the third enzyme provides nucleic acid molecules comprising a sequence of interest truncated in 3′.

In step e) of the method of the invention, cleaved nucleic acid molecules obtained from step d) are circularized by intra-molecular ligation. This ligation may be performed as disclosed above.

Circularized nucleic acid molecules obtained from step e) comprise, in the 5′ end to 3′ end direction, a truncated sequence of interest, a reverse priming site, a forward priming site and a barcode sequence which is between the forward priming site and the sequence of interest or between the sequence of interest and the reverse priming site.

In one embodiment, the barcode sequence is located between the forward priming site and the sequence of interest. In this embodiment, the sequence of interest comprised in said circularized nucleic acid molecules is truncated in 5′.

In another embodiment, the barcode sequence is located between the reverse priming site and the sequence of interest. In this embodiment, the sequence of interest comprised in said circularized nucleic acid molecules is truncated in 3′.

Nucleic acid molecules obtained after steps a), b), c), d) and e) comprise a sequence of interest truncated in 5′ or 3′ according to the position of the barcode (upstream or downstream to the sequence of interest). The method of the invention may further comprise additional steps in order to truncate the other end of the sequence of interest thereby providing nucleic acid molecules comprising a sequence of interest truncated in 3′ and 5′.

In one embodiment, linear nucleic acid molecules provided in step a) comprise the sequence of interest at one of its end (e.g., FIG. 1a, FIG. 1d, FIG. 1g and FIG. 1h; FIG. 2a, FIG. 2d, FIG. 2g and FIG. 2h; FIGS. 3a and 3d) and the method further comprises a step a′) of cleaving the nucleic acid molecules, after step a) and before step b), thereby providing a truncated sequence of interest. Preferably, the cleavage is performed by using a sequence independent technique of cleavage (SITC), such as sonication. In particular, if the sequence of interest is at the 3′ end of the linear nucleic acid molecules provided in step a) (e.g., FIG. 1a and FIG. 1h; FIG. 2a and FIG. 2h; FIG. 3a), the obtained nucleic acid molecules present a 3′ truncated sequence of interest. Alternatively, if the sequence of interest is at the 5′ end of the linear nucleic acid molecules provided in step a) (e.g., FIG. 1d and FIG. 1g; FIG. 2d and FIG. 2g; 3d), the obtained nucleic acid molecules present a 5′ truncated sequence of interest. Cleaved molecules may be separated by electrophoresis and molecules of the desired size may be purified from the electrophoresis gel.

In another embodiment, linear nucleic acid molecules provided in step a) do not comprise the sequence of interest at one of its end but comprise a recognition site for a fourth restriction enzyme located at the end of the sequence of interest that is opposite to the end adjacent to the barcode. In this embodiment, the method further comprises after step e),

f) digesting circular nucleic acid molecules obtained from step e) with the fourth restriction enzyme, thereby providing linear nucleic acid molecules comprising the non truncated end of the sequence of interest at one of its end;

g) cleaving digested nucleic acid molecules obtained from step f) in the sequence of interest, thereby providing linear nucleic acid molecules comprising a sequence of interest truncated in 5′ and 3′.

Cleaved molecules may be separated by electrophoresis and molecules of the desired size may be purified from the electrophoresis gel.

The method of invention may further comprise a step h) of circularizing nucleic acid molecules obtained from step g) by intra-molecular ligation. This ligation may be performed as disclosed above.

According to the structure of the nucleic acid molecules providing in step a), the circular molecules obtained from step h) comprise, in the 5′ to 3′ direction:
a forward priming site, a barcode sequence, a sequence of interest truncated in 3′ and 5′, a recognition site for a third restriction enzyme, a reverse priming site and a curvature module comprising a recognition site for a second restriction enzyme, or

a forward priming site, a sequence of interest truncated in 5′ and in 3′, a barcode sequence, a reverse priming site and a curvature module comprising a recognition site for a second restriction enzyme.

In a preferred embodiment, the method of the invention comprises
providing a set or a plurality of sets of linear nucleic acid molecules, each nucleic acid molecule comprising from 5′ end to 3′ end a recognition site for a third restriction enzyme, a reverse priming site, a curvature module comprising a recognition site for a second restriction enzyme, a forward priming site, a barcode sequence, a recognition site for a first restriction enzyme, optionally a universal priming site, and a sequence of interest (FIG. 3a) (step a);
cleaving said linear nucleic acid molecules by using a sequence independent technique of cleavage such as sonication, thereby providing linear nucleic acid molecules comprising from 5′ end to 3′ end, a recognition site for a third restriction enzyme, a reverse priming site, a curvature module comprising a recognition site for a second restriction enzyme, a forward priming site, a barcode sequence, optionally a universal priming site, and a sequence of interest truncated in 3′;
circularizing said linear nucleic acid molecules by intra-molecular ligation, thereby providing circular nucleic acid molecules comprising, in the 5′ to 3′ direction, a barcode sequence, optionally a universal priming site, a sequence of interest truncated in 3′, a recognition site for a third restriction enzyme, a reverse priming site, a curvature module comprising a recognition site for a second restriction enzyme, a forward priming site (step b);
digesting said circular nucleic acid molecules with the first restriction enzyme, thereby providing linear nucleic acid molecules comprising from 5′ end to 3′ end, optionally a universal priming site, a sequence of interest truncated in 3′, a recognition site for a third restriction enzyme, a reverse priming site, a curvature module comprising a recognition site for a second restriction enzyme, a forward priming site and a barcode sequence (step c);
cleaving said digested nucleic acid molecules by digestion with the third restriction enzyme, thereby providing linear nucleic acid molecules comprising from 5′ end to 3′ end, a sequence of interest truncated in 3′ and 5′, a recognition site for a third restriction enzyme, a reverse priming site, a curvature module comprising a recognition site for a second restriction enzyme, a forward priming site and a barcode sequence (step d); and
circularizing said cleaved nucleic acid molecules by intra-molecular ligation, thereby providing circular nucleic acid molecules comprising, in the 5′ to 3′ direction, a forward priming site, a barcode sequence, a sequence of interest truncated in 3′ and 5′, a recognition site for a third restriction enzyme, a reverse priming site and a curvature module comprising a recognition site for a second restriction enzyme (step e).

In another preferred embodiment, the method of the invention comprises
providing a set or a plurality of sets of linear nucleic acid molecules, each nucleic acid molecule comprising from 5′ end to 3′ end, a reverse priming site, a curvature module comprising a recognition site for a second restriction enzyme, a forward priming site, a barcode sequence, a recognition site for a first restriction enzyme, optionally a universal priming site, and a sequence of interest (FIG. 3a) (step a);
cleaving said linear nucleic acid molecules by using a sequence independent technique of cleavage such as sonication, thereby providing linear nucleic acid molecules comprising from 5′ end to 3′ end, a reverse priming site, a curvature module comprising a recognition site for a second restriction enzyme, a forward priming site, a barcode sequence, optionally a universal priming site, and a sequence of interest truncated in 3′;
circularizing said linear nucleic acid molecules by intra-molecular ligation, thereby providing circular nucleic acid molecules comprising, in the 5′ to 3′ direction, a barcode sequence, optionally a universal priming site, a sequence of interest truncated in 3′, a reverse priming site, a curvature module comprising a recognition site for a second restriction enzyme and a forward priming site (step b);
digesting said circular nucleic acid molecules with the first restriction enzyme, thereby providing linear nucleic acid molecules comprising from 5′ end to 3′ end, optionally a universal priming site, a sequence of interest truncated in 3′, a reverse priming site, a curvature module comprising a recognition site for a second restriction enzyme, a forward priming site and a barcode sequence (step c);
cleaving said digested nucleic acid molecules by using a sequence independent technique such as sonication, thereby providing linear nucleic acid molecules comprising from 5′ end to 3′ end, a sequence of interest truncated in 3′ and 5′, a reverse priming site, a curvature module comprising a recognition site for a second restriction enzyme, a forward priming site and a barcode sequence (step d); and
circularizing said cleaved nucleic acid molecules by intra-molecular ligation, thereby providing circular nucleic acid molecules comprising, in the 5′ to 3′ direction, a forward priming site, a barcode sequence, a sequence of interest truncated in 3′ and 5′, a reverse priming site and a curvature module comprising a recognition site for a second restriction enzyme (step e).

In another preferred embodiment, the method of the invention comprises
providing a set or a plurality of sets of linear nucleic acid molecules, each nucleic acid molecule comprising from 5′ end to 3′ end, a sequence of interest, optionally a universal priming site, a recognition site for a first restriction enzyme, a barcode sequence, a reverse priming site, a curvature module comprising a recognition site for a second restriction enzyme, a forward priming site and a recognition site for a third restriction enzyme (FIG. 3d) (step a);
cleaving said linear nucleic acid molecules by using a sequence independent technique of cleavage such as sonication, thereby providing linear nucleic acid molecules comprising from 5′ end to 3′ end, a sequence of interest truncated in 5′, optionally a universal priming site, a recognition site for a first restriction enzyme, a barcode sequence, a reverse priming site, a curvature module comprising a recognition site for a second restriction enzyme, a forward priming site and a recognition site for a third restriction enzyme;
circularizing said linear nucleic acid molecules by intra-molecular ligation, thereby providing circular nucleic acid molecules comprising, in the 5′ to 3′ direction, a barcode sequence, a reverse priming site, a curvature module comprising a recognition site for a second restriction enzyme, a forward priming site, a recognition site for a third restriction enzyme, a sequence of interest truncated in 5′ and, optionally, a universal priming site (step b);
digesting said circular nucleic acid molecules with the first restriction enzyme, thereby providing linear nucleic acid molecules comprising from 5′ end to 3′ end, a barcode sequence, a reverse priming site, a curvature module comprising a recognition site for a second restriction enzyme, a forward priming site, a recognition site for a third restriction enzyme, a sequence of interest truncated in 5′ and, optionally, a universal priming site (step c);
cleaving said digested nucleic acid molecules by digestion with the third restriction enzyme, thereby providing linear nucleic acid molecules comprising from 5′ end to 3′ end, a barcode sequence, a reverse priming site, a curvature module comprising a recognition site for a second restriction enzyme, a forward priming site, a recognition site for a third restriction enzyme, a sequence of interest truncated in 5′ and in 3′ (step d); and
circularizing said cleaved nucleic acid molecules by intra-molecular ligation, thereby providing circular nucleic acid molecules comprising, in the 5′ to 3′ direction, a forward priming site, a recognition site for a third restriction enzyme, a sequence of interest truncated in 3′ and 5′, a barcode sequence, a reverse priming site and a curvature module comprising a recognition site for a second restriction enzyme (step e).

In another preferred embodiment, the method of the invention comprises
providing a set or a plurality of sets of linear nucleic acid molecules, each nucleic acid molecule comprising from 5′ end to 3′ end, a sequence of interest, optionally a universal priming site, a recognition site for a first restriction enzyme, a barcode sequence, a reverse priming site, a curvature module comprising a recognition site for a second restriction enzyme and a forward priming site (FIG. 3d);
cleaving said linear nucleic acid molecules by using a sequence independent technique of cleavage such as sonication, thereby providing linear nucleic acid molecules comprising from 5′ end to 3′ end, a sequence of interest truncated in 5′, optionally a universal priming site, a recognition site for a first restriction enzyme, a barcode sequence, a reverse priming site, a curvature module comprising a recognition site for a second restriction enzyme and a forward priming site;
circularizing said linear nucleic acid molecules by intra-molecular ligation, thereby providing circular nucleic acid molecules comprising, in the 5′ to 3′ direction, a barcode sequence, a reverse priming site, a curvature module comprising a recognition site for a second restriction enzyme, a forward priming site, a sequence of interest truncated in 5′, optionally a universal priming site, and a recognition site for a first restriction enzyme (step b);
digesting said circular nucleic acid molecules with the first restriction enzyme, thereby providing linear nucleic acid molecules comprising from 5′ end to 3′ end, a barcode sequence, a reverse priming site, a curvature module comprising a recognition site for a second restriction enzyme, a forward priming site, a sequence of interest truncated in 5′ and, optionally, a universal priming site (step c);
cleaving said digested nucleic acid molecules by using a sequence independent technique such as sonication, thereby providing linear nucleic acid molecules comprising from 5′ end to 3′ end, a barcode sequence, a reverse priming site, a curvature module comprising a recognition site for a second restriction enzyme, a forward priming site, a sequence of interest truncated in 5′ and in 3′ (step d); and
circularizing said cleaved nucleic acid molecules by intra-molecular ligation, thereby providing circular nucleic acid molecules comprising, in the 5′ to 3′ direction, a forward priming site, a sequence of interest truncated in 5′ and in 3′, a barcode sequence, a reverse priming site and a curvature module comprising a recognition site for a second restriction enzyme (step e).

In a preferred embodiment, the method of the invention comprises
providing a set or a plurality of sets of linear nucleic acid molecules, each nucleic acid molecule comprising from 5′ end to 3′ end, a barcode sequence, a recognition site for a first restriction enzyme, a sequence of interest, optionally a universal priming site, a recognition site for a third restriction enzyme and a recognition site for a fourth restriction enzyme, a reverse priming site, a curvature module comprising a recognition site for a second restriction enzyme, and a forward priming site (FIG. 3b) (step a);
circularizing said linear nucleic acid molecules by intra-molecular ligation, thereby providing circular nucleic acid molecules comprising, in the 5′ to 3′ direction, a barcode sequence, a recognition site for a first restriction enzyme, a sequence of interest, optionally a universal priming site, a recognition site for a third restriction enzyme and a recognition site for a fourth enzyme, a reverse priming site, a curvature module comprising a recognition site for a second restriction enzyme, and a forward priming site (step b);
digesting said circular nucleic acid molecules with the first restriction enzyme, thereby providing linear nucleic acid molecules comprising from 5′ end to 3′ end, a sequence of interest, optionally a universal priming site, a recognition site for a third restriction enzyme and a recognition site for a fourth enzyme, a reverse priming site, a curvature module comprising a recognition site for a second restriction enzyme, a forward priming site and a barcode sequence (step c);
cleaving said digested nucleic acid molecules by digestion with the third restriction enzyme, thereby providing linear nucleic acid molecules comprising from 5′ end to 3′ end, a sequence of interest truncated in 5′, optionally a universal priming site, a recognition site for a third restriction enzyme and a recognition site for a fourth restriction enzyme, a reverse priming site, a curvature module comprising a recognition site for a second restriction enzyme, a forward priming site and a barcode sequence (step d);
circularizing said cleaved nucleic acid molecules by intra-molecular ligation, thereby providing circular nucleic acid molecules comprising, in the 5′ to 3′ direction, a forward priming site, a barcode sequence, a sequence of interest truncated in 5′, optionally a universal priming site, a recognition site for a third restriction enzyme and a recognition site for a fourth restriction enzyme, a reverse priming site, a curvature module comprising a recognition site for a second restriction enzyme (step e),
digesting said circular nucleic acid molecules with the fourth restriction enzyme, thereby providing linear nucleic acid molecules comprising from 5′ end to 3′ end, a reverse priming site, a curvature module comprising a recognition site for a second restriction enzyme, a forward priming site, a barcode sequence, a sequence of interest truncated in 5′ and, optionally, a universal priming site (step f);
cleaving said linear nucleic acid by using a sequence independent technique such as sonication, thereby providing linear nucleic acid molecules comprising from 5′ end to 3′ end, a reverse priming site, a curvature module comprising a recognition site for a second restriction enzyme, a forward priming site, a barcode sequence, a sequence of interest truncated in 5′ and 3′ (step g); and
circularizing said cleaved nucleic acid molecules by intra-molecular ligation, thereby providing circular nucleic acid molecules comprising, in the 5′ to 3′ direction, a forward priming site, a barcode sequence, a sequence of interest truncated in 5′ and in 3′, a reverse priming site and a curvature module comprising a recognition site for a second restriction enzyme (step h).

In a preferred embodiment, the method of the invention comprises
providing a set or a plurality of sets of linear nucleic acid molecules, each nucleic acid molecule comprising from 5′ end to 3′ end, a barcode sequence, a recognition site for a first restriction enzyme, a sequence of interest, optionally a universal priming site, a recognition site for a fourth enzyme, a reverse priming site, a curvature module comprising a recognition site for a second restriction enzyme, and a forward priming site (FIG. 3b) (step a);
circularizing said linear nucleic acid molecules by intra-molecular ligation, thereby providing circular nucleic acid molecules comprising, in the 5′ to 3′ direction, a barcode sequence, a recognition site for a first restriction enzyme, a sequence of interest, optionally a universal priming site, a recognition site for a fourth enzyme, a reverse priming site, a curvature module comprising a recognition site for a second restriction enzyme, and a forward priming site (step b);
digesting said circular nucleic acid molecules with the first restriction enzyme, thereby providing linear nucleic acid molecules comprising from 5′ end to 3′ end, a sequence of interest, optionally a universal priming site, a recognition site for a fourth enzyme, a reverse priming site, a curvature module comprising a recognition site for a second restriction enzyme, a forward priming site and a barcode sequence (step c);
cleaving said digested nucleic acid molecules by using a sequence independent technique such as sonication, thereby providing linear nucleic acid molecules comprising from 5′ end to 3′ end, a sequence of interest truncated in 5′, optionally a universal priming site, a recognition site for a fourth restriction enzyme, a reverse priming site, a curvature module comprising a recognition site for a second restriction enzyme, a forward priming site and a barcode sequence (step d);
circularizing said cleaved nucleic acid molecules by intra-molecular ligation, thereby providing circular nucleic acid molecules comprising, in the 5′ to 3′ direction, a forward priming site, a barcode sequence, a sequence of interest truncated in 5′, optionally a universal priming site, a recognition site for a fourth restriction enzyme, a reverse priming site, a curvature module comprising a recognition site for a second restriction enzyme (step e),
digesting said circular nucleic acid molecules with the fourth restriction enzyme, thereby providing linear nucleic acid molecules comprising from 5′ end to 3′ end, a reverse priming site, a curvature module comprising a recognition site for a second restriction enzyme, a forward priming site, a barcode sequence, a sequence of interest truncated in 5′ and, optionally, a universal priming site (step 1);
cleaving said linear nucleic acid by using a sequence independent technique such as sonication, thereby providing linear nucleic acid molecules comprising from 5′ end to 3′ end, a reverse priming site, a curvature module comprising a recognition site for a second restriction enzyme, a forward priming site, a barcode sequence, a sequence of interest truncated in 5′ and 3′ (step g); and
circularizing said cleaved nucleic acid molecules by intra-molecular ligation, thereby providing circular nucleic acid molecules comprising, in the 5′ to 3′ direction, a forward priming site, a barcode sequence, a sequence of interest truncated in 5′ and in 3′, a reverse priming site and a curvature module comprising a recognition site for a second restriction enzyme (step h).

In a preferred embodiment, the method of the invention comprises
providing a set or a plurality of sets of linear nucleic acid molecules, each nucleic acid molecule comprising from 5′ end to 3′ end, a reverse priming site, a curvature module comprising a recognition site for a second restriction enzyme, a forward priming site, a recognition site for a third restriction enzyme and a recognition site for a fourth enzyme, optionally a universal priming site, a sequence of interest, a recognition site for a first restriction enzyme and a barcode sequence (FIG. 3c) (step a);
circularizing said linear nucleic acid molecules by intra-molecular ligation, thereby providing circular nucleic acid molecules comprising, in the 5′ to 3′ direction, a forward priming site, a recognition site for a third restriction enzyme and a recognition site for a fourth enzyme, optionally a universal priming site, a sequence of interest, a recognition site for a first restriction enzyme, a barcode sequence, a reverse priming site and a curvature module comprising a recognition site for a second restriction enzyme (step b);
digesting said circular nucleic acid molecules with the first restriction enzyme, thereby providing linear nucleic acid molecules comprising from 5′ end to 3′ end, a barcode sequence, a reverse priming site, a curvature module comprising a recognition site for a second restriction enzyme, a forward priming site, a recognition site for a third restriction enzyme and a recognition site for a fourth enzyme, optionally a universal priming site and a sequence of interest (step c);
cleaving said digested nucleic acid molecules by digestion with the third restriction enzyme, thereby providing linear nucleic acid molecules comprising from 5′ end to 3′ end, a barcode sequence, a reverse priming site, a curvature module comprising a recognition site for a second restriction enzyme, a forward priming site, a recognition site for a third restriction enzyme and a recognition site for a fourth enzyme, optionally a universal priming site and a sequence of interest truncated in 3′ (step d);
circularizing said cleaved nucleic acid molecules by intra-molecular ligation, thereby providing circular nucleic acid molecules comprising, in the 5′ to 3′ direction, a forward priming site, a recognition site for a third restriction enzyme and a recognition site for a fourth enzyme, optionally a universal priming site, a sequence of interest truncated in 3′, a barcode sequence, a reverse priming site and a curvature module comprising a recognition site for a second restriction enzyme (step e),

digesting said circular nucleic acid molecules with the fourth restriction enzyme, thereby providing linear nucleic acid molecules comprising from 5′ end to 3′ end, optionally a universal priming site, a sequence of interest truncated in 3′, a barcode sequence, a reverse priming site, a curvature module comprising a recognition site for a second restriction enzyme and a forward priming site (step f);

cleaving said linear nucleic acid by using a sequence independent technique such as sonication, thereby providing linear nucleic acid molecules comprising from 5′ end to 3′ end, a sequence of interest truncated in 5′ and 3′, a barcode sequence, a reverse priming site, a curvature module comprising a recognition site for a second restriction enzyme and a forward priming site (step g); and
circularizing said cleaved nucleic acid molecules by intra-molecular ligation, thereby providing circular nucleic acid molecules comprising, in the 5′ to 3′ direction, a forward priming site, a sequence of interest truncated in 5′ and in 3′, a barcode sequence, a reverse priming site and a curvature module comprising a recognition site for a second restriction enzyme (step h).

In a preferred embodiment, the method of the invention comprises
providing a set or a plurality of sets of linear nucleic acid molecules, each nucleic acid molecule comprising from 5′ end to 3′ end, a reverse priming site, a curvature module comprising a recognition site for a second restriction enzyme, a forward priming site, a recognition site for a fourth enzyme, optionally a universal priming site, a sequence of interest, a recognition site for a first restriction enzyme and a barcode sequence (FIG. 3c) (step a);
circularizing said linear nucleic acid molecules by intra-molecular ligation, thereby providing circular nucleic acid molecules comprising, in the 5′ to 3′ direction, a forward priming site, a recognition site for a fourth enzyme, optionally a universal priming site, a sequence of interest, a recognition site for a first restriction enzyme, a barcode sequence, a reverse priming site and a curvature module comprising a recognition site for a second restriction enzyme (step b);
digesting said circular nucleic acid molecules with the first restriction enzyme, thereby providing linear nucleic acid molecules comprising from 5′ end to 3′ end, a barcode sequence, a reverse priming site, a curvature module comprising a recognition site for a second restriction enzyme, a forward priming site, a recognition site for a fourth enzyme, optionally a universal priming site and a sequence of interest (step c);
cleaving said digested nucleic acid molecules by using a sequence independent technique such as sonication, thereby providing linear nucleic acid molecules comprising from 5′ end to 3′ end, a barcode sequence, a reverse priming site, a curvature module comprising a recognition site for a second restriction enzyme, a forward priming site, a recognition site for a fourth enzyme, optionally a universal priming site and a sequence of interest truncated in 3′ (step d);
circularizing said cleaved nucleic acid molecules by intra-molecular ligation, thereby providing circular nucleic acid molecules comprising, in the 5′ to 3′ direction, a forward priming site, a recognition site for a fourth enzyme, optionally a universal priming site, a sequence of interest truncated in 3′, a barcode sequence, a reverse priming site and a curvature module comprising a recognition site for a second restriction enzyme (step e),
digesting said circular nucleic acid molecules with the fourth restriction enzyme, thereby providing linear nucleic acid molecules comprising from 5′ end to 3′ end, optionally a universal priming site, a sequence of interest truncated in 3′, a barcode sequence, a reverse priming site, a curvature module comprising a recognition site for a second restriction enzyme and a forward priming site (step f);
cleaving said linear nucleic acid by using a sequence independent technique such as sonication, thereby providing linear nucleic acid molecules comprising from 5′ end to 3′ end, a sequence of interest truncated in 5′ and 3′, a barcode sequence, a reverse priming site, a curvature module comprising a recognition site for a second restriction enzyme and a forward priming site (step g); and
circularizing said cleaved nucleic acid molecules by intra-molecular ligation, thereby providing circular nucleic acid molecules comprising, in the 5′ to 3′ direction, a forward priming site, a sequence of interest truncated in 5′ and in 3′, a barcode sequence, a reverse priming site and a curvature module comprising a recognition site for a second restriction enzyme (step h).

In an embodiment, the nucleic acid molecules provided in step a) are attached to a first member of an affinity binding pair and the method further comprises one or several steps of binding nucleic acid molecules on a solid support through the interaction between the first member of an affinity binding pair attached to said nucleic acid molecules and second members of said affinity binding pair linked to the solid support. Useful solid supports include any rigid or semi-rigid surface on which a member of an affinity binding pair may be linked. The support can be any porous or non-porous water insoluble material, including, without limitation, membranes, filters, chips, magnetic or nonmagnetic beads, and polymers. Preferably, the solid support is selected from silica-based membranes and beads. More preferably, the solid support is beads and more particularly polystyrene beads. These beads may have a diameter from 1 to 10 micrometer. In a particular embodiment, the nucleic acid molecules provided in step a) are attached to biotin and are bound to a solid support, preferably magnetic beads, coated with streptavidin. Nucleic acid molecules may be bound to a solid support before a circularization step by intra-molecular ligation in order to prevent intermolecular events or before each change of reaction mix in order to facilitate the recovering of nucleic acid molecules. The amount of solid support may be easily adjusted by the skilled person according to the concentration of nucleic acid molecules.

In a preferred embodiment, linear nucleic acid molecules obtained from step g) are bound to a solid support before circularization of step h).

In another embodiment, circular nucleic acid molecules obtained from step h) are then bound to a solid support.

Circular nucleic acid molecules obtained from step h) may be digested by the second restriction enzyme, thereby providing linear nucleic acid molecules comprising from 5′ end to 3′ end,
a forward priming site, a barcode sequence, a sequence of interest truncated in 5′ and 3′ and a reverse priming sequence, or
a forward priming site, a sequence of interest truncated in 5′ and 3′, a barcode sequence and a reverse priming sequence.

If the nucleic acid molecules comprise a curvature module comprising the recognition site for the second restriction enzyme, nucleic acid molecules obtained by digestion with the second restriction enzyme comprises at each end, a part of the curvature module.

Preferably, nucleic acid molecules are bound on a solid support prior to be digested with the second restriction enzyme.

Nucleic acid molecules digested with the second restriction enzyme may then be amplified, for example by PCR or by any other method known by the skilled person. Preferably, primers used for this amplification comprise:
a forward primer hybridizing to the forward priming site, and
a reverse primer hybridizing to the reverse priming site.

The product of this amplification may then be sequenced by using any high-throughput sequencing plateform.

The method may further comprise one or several steps of repairing ends of cleaved or digested nucleic acid molecules. Preferably, ends of nucleic acid molecules are repaired after each step of cleavage or of digestion and/or each step of circularization. Repairing may comprise restoring ends and/or phosphorylating ends. Restoration and phosphorylation may be performed by any method known by the skilled person. For example, restoration may be performed by using a specific DNA polymerase, such as T4 DNA polymerase, in presence of dNTP, and phosphorylation may be performed by using a DNA kinase, such as T4 polynucleotide kinase, in presence of ATP.

Optionally, the method further comprises one or several steps of degrading linear nucleic acid molecules with an ATP-Dependent DNase that selectively hydrolyzes linear double-stranded nucleic acid molecules. The Plasmid-Safe™ ATP-Dependent DNase (Epicentre) ATP-Dependent DNase may be selected to degrade specifically linear DNA. It is particularly indicated to degrade linear nucleic acid molecules after a step of circularization, preferably after each step of circularization.

The present invention concerns kits suitable for preparing the nucleic acid molecules provided at the step a) of the method disclosed herein. In particular, said kits may comprise any forward or reverse primer or a set thereof as disclosed previously for preparing the nucleic acid molecules provided in step a).

The kit may comprise at least one first forward primer comprising, from its 5′ end to its 3′ end,
a reverse priming site, a recognition site for a second restriction enzyme, and a forward priming site or a part thereof including its 5′ end; or
a reverse priming site, a curvature module comprising a recognition site for a second restriction enzyme, and a forward priming site or a part thereof including its 5′ end.

By “a part thereof” is intended at least 10, 15 or 20 nucleotides thereof.

In a first embodiment, the kit further comprises at least one second forward primer comprising, from its 5′ end to its 3′ end, a forward priming site or a part thereof including its 3′ end and overlapping the part of forward priming site of the first forward primer, a barcode sequence, a recognition site for a first restriction enzyme and either at least 10 nucleotides of the 5′ end of the sequence of interest or a universal priming site. Preferably, the at least one second forward primer comprises, from its 5′ end to its 3′ end, a forward priming site or a part thereof including its 3′ end and overlapping the part of forward priming site of the first forward primer, a barcode sequence, a recognition site for a first restriction enzyme and a universal priming site. More preferably, the kit comprises one first forward primer and several second forward primers differing by their barcode sequences and, if present, by the at least 10 nucleotides of the 5′ end of the sequence of interest. Indeed, each second forward primer is designed to be specific to a different sequence of interest. In particular, if between 1 and 1,000 different sequences of interest are considered, 1 and 1000 different second forward primers are designed, each including a distinct barcode to be respectively associated to each sequence of interest, the other element remaining the same (e.g., the forward priming site or the part thereof, the recognition site for the first restriction enzyme and the universal priming site).

In a second embodiment, the at least one first forward primer further comprises, at its 3′ end, a barcode sequence, a recognition site for a first restriction enzyme and either at least 10 nucleotides of the 5′ end of the sequence of interest or a universal priming site. Preferably, the at least one first forward primer further comprises, at its 3′ end, a barcode sequence, a recognition site for a first restriction enzyme and a universal priming site. More preferably, the at least one first forward primer further comprises, at its 5′ end, a recognition site for the third restriction enzyme and/or a recognition site for the fourth restriction enzyme. Despite the kit may comprise one first forward primer, the kit preferably comprises several first forward primers differing by their barcode sequences and, if present, by the at least 10 nucleotides of the 5′ end of the sequence of interest, the other elements remaining the same (i.e., if present, the reverse priming site, the curvature module, the recognition site for the second restriction enzyme, the forward priming site or the part thereof, the universal priming site).

According to the first or second embodiment, the kit may further comprise one reverse primer comprising at least 10 nucleotides of the 3′ end of the sequence of interest or several reverse primers, each comprising at least 10 nucleotides of the 3′ end of a different sequence of interest.

In a third embodiment, the at least one first forward primer further comprises at its 3′ end a recognition site for the third restriction enzyme and/or a recognition site for the fourth restriction enzyme, and either at least 10 nucleotides of the 5′ end of the sequence of interest or a universal priming site. Preferably, the at least one first forward primer further comprises at its 3′ end a recognition site for the third restriction enzyme and/or a recognition site for the fourth restriction enzyme, and a universal priming site. Accordingly, the kit may comprise one first forward primer. In this embodiment, the kit may further comprise a reverse primer comprising, from its 5′ end to its 3′ end, a barcode sequence, a recognition site for a first restriction enzyme and at least 10 nucleotides of the 3′ end of the sequence of interest. Preferably, the kit further comprises several reverse primers, differing by their barcode sequences and by the at least 10 nucleotides of the 3′ end of the sequence of interest.

According to the first, second or third embodiment, the kit may further comprise at least one forward primer comprising, from its 5′ end to its 3′ end, a universal priming site and at least 10 nucleotides of the 5′ end of the sequence of interest. In particular, the kit may further comprise several forward primers comprising, from its 5′ end to its 3′ end, a universal priming site and at least 10 nucleotides of the 5′ end of one of the different sequences of interest.

Alternatively, the kit may comprise at least one first reverse primer comprising, from its 5′ end to its 3′ end,
a forward priming site, a recognition site for a second restriction enzyme, and a reverse priming site or a part thereof including its 3′ end; or
a forward priming site, a curvature module comprising a recognition site for the second restriction enzyme, and a reverse priming site or a part thereof including its 3′ end.

By “a part thereof” is intended at least 10, 15 or 20 nucleotides thereof.

In a first embodiment, the kit further comprises at least one second reverse primer comprising, from its 5′ end to its 3′ end, a reverse priming site or a part thereof including its 5′ end and overlapping the part of reverse priming site of the first reverse primer, a barcode sequence, a recognition site for a first restriction enzyme and either at least 10 nucleotides of the 3′ end of the sequence of interest or a universal priming site. Preferably, the at least one second reverse primer comprises, from its 5′ end to its 3′ end, a reverse priming site or a part thereof including its 5′ end and overlapping the part of reverse priming site of the first reverse primer, a barcode sequence, a recognition site for a first restriction enzyme and a universal priming site. More preferably, the kit comprises one first reverse primer and several second reverse primers differing by their barcode sequences and, if present, by the at least 10 nucleotides of the 3′ end of the sequence of interest. Indeed, each second reverse primer is designed to be specific to a different sequence of interest. In particular, if between 2 and 1,000 different sequences of interest are considered, 2 and 1,000 different second reverse primers are designed, each including a distinct barcode to be associated to each sequence of interest, the other element remaining the same (e.g., the reverse priming site or the part thereof, the recognition site for the first restriction enzyme and the universal priming site).

In a second embodiment, the at least one first reverse primer further comprises, at its 3′ end, a barcode sequence, a recognition site for a first restriction enzyme and either at least 10 nucleotides of the 3′ end of the sequence of interest or a universal priming site. Preferably, the at least one first reverse primer further comprises, at its 3′ end, a barcode sequence, a recognition site for a first restriction enzyme and a universal priming site. More preferably the at least one first reverse primer further comprises, at its 5′ end, a recognition site for the third restriction enzyme and/or a recognition site for the fourth restriction enzyme. Despite the kit may comprise one first reverse primer, the kit preferably comprises several first reverse primers differing by their barcode sequences and, if present, by the at least 10 nucleotides of the 3′ end of the sequence of interest, the other elements remaining the same (i.e., if present, the reverse priming site, the curvature module, the recognition site for the second restriction enzyme, the forward priming site or the part thereof, the universal priming site).

According to the first or second embodiment, the kit may further comprise one forward primer comprising at least 10 nucleotides of the 5′ end of the sequence of interest or several forward primers, each comprising at least 10 nucleotides of the 5′ end of a different sequence of interest.

In a third embodiment, the at least one first reverse primer further comprises at its 3′ end a recognition site for the third restriction enzyme and/or a recognition site for the fourth restriction enzyme, and either at least 10 nucleotides of the 3′ end of the sequence of interest or a universal priming site. Preferably, the at least one first reverse primer further comprises at its 3′ end a recognition site for the third restriction enzyme and/or a recognition site for the fourth restriction enzyme and a universal priming site. Accordingly, the kit may comprise one first reverse primer. In this embodiment, the kit may further comprise a forward primer comprising, from its 5′ end to its 3′ end, a barcode sequence, a recognition site for a first restriction enzyme and at least 10 nucleotides of the 5′ end of the sequence of interest. Preferably, the kit further comprises several forward primers, differing by their barcode sequences and by the at least 10 nucleotides of the 5′ end of the sequence of interest.

According to the first, second or third embodiment, the kit may further comprise at least one reverse primer comprising, from its 5′ end to its 3′ end, a universal priming site and at least 10 nucleotides of the 3′ end of the sequence of interest. In particular, the kit may further comprise several reverse primers comprising, from its 5′ end to its 3′ end, a universal priming site and at least 10 nucleotides of the 3′ end of one of the different sequences of interest.

In a particular embodiment, the kit comprises at least one forward primer for each sequence of interest to be sequenced, comprising, from its 5′ end to its 3′ end:
a) a reverse priming site, a recognition site for the second restriction enzyme, a forward priming site, a barcode sequence, a recognition site for the first restriction enzyme and at least 10 nucleotides of the 3′ end of the sequence of interest; or
b) a reverse priming site, a curvature module comprising a recognition site for the second restriction enzyme, a forward priming site, a barcode sequence, a recognition site for the first restriction enzyme and at least 10 nucleotides of the 3′ end of the sequence of interest; or
c) a forward priming site, a recognition site for the second restriction enzyme, a reverse priming site and at least 10 nucleotides of the 5′ end of the sequence of interest; or
d) a reverse priming site, a curvature module optionally attached to biotin and comprising a recognition site for the second restriction enzyme, a forward priming site and at least 10 nucleotides of the 5′ end of the sequence of interest.

Optionally, the kit may also comprise the appropriate or associated reverse primer(s). In particular, the kit may include a reverse primer specific of the 3′ end of the sequence of interest for the forward primers of type a) and b); or a reverse primer comprising, from its 5′end to its 3′ end, a barcode sequence, a recognition site for the first restriction enzyme and at least 10 nucleotides of the 3′ end of the sequence of interest for the forward primers of type c) and d).

The kit may comprise one forward primer and its associated reverse primer. However, preferably, the kit comprises several forward primers and optionally associated reverse primers, each designed to be specific to a different sequence of interest. The forward and reverse primers differ from each other by their barcode sequence and by the at least 10 nucleotides of the 3′ end of the specific sequence of interest, the other elements (e.g., the forward priming site, the recognition site for the second restriction enzyme, the reverse priming site, the curvature module and the recognition site for the first restriction enzyme remaining the same).

In another particular embodiment, the kit comprises at least one reverse primer for each sequence of interest to be sequenced, comprising, from its 5′ end to its 3′ end,
a) a forward priming site, a recognition site for the second restriction enzyme, a reverse priming site, a barcode sequence, a recognition site for the first restriction enzyme and at least 10 nucleotides of the 3′ end of the sequence of interest; or
b) a forward priming site, a curvature module comprising a recognition site for the second restriction enzyme, a reverse priming site, a barcode sequence, a recognition site for the first restriction enzyme and at least 10 nucleotides of the 3′ end of the sequence of interest;
c) a forward priming site, a recognition site for the second restriction enzyme, a reverse priming site and at least 10 nucleotides of the 3′ end of the sequence of interest; or
d) a forward priming site, a curvature module optionally attached to biotin and comprising a recognition site for the second restriction enzyme, a reverse priming site and at least 10 nucleotides of the 3′ end of the sequence of interest.

Optionally, the kit may also comprise the appropriate or associated forward primer(s). In particular, the kit may include a forward primer specific of the 5′ end of the sequence of interest for the reverse primers of type a) and b); or a forward primer comprising, from its 5′end to its 3′ end, a barcode sequence, a recognition site for the first restriction enzyme and at least 10 nucleotides of the 5′ end of the sequence of interest for the reverse primers of type c) and d).

The kit may comprise one reverse primer and its associated forward primer. However, preferably, the kit comprises several reverse primers and optionally associated forward primers, each designed to be specific to a different sequence of interest. The forward and reverse primers differ from each other by their barcode sequence and by the at least 10 nucleotides of the 3′ end of the specific sequence of interest, the other elements (e.g., if present, the forward priming site, the recognition site for the first, second, third and fourth restriction enzyme, the reverse priming site and the curvature module remaining the same).

In a preferred embodiment of the kit, the first forward primer or first reverse primer comprises a curvature module comprising a recognition site for the second restriction enzyme. Preferably, the curvature module is attached to a first member of an affinity binding pair, preferably biotin. In particular, the curvature module may be as detailed below. Preferably, it comprises or consists of the sequence selected from the group consisting of SEQ ID No. 1 and SEQ ID No. 2.

In addition to the primers, the kits may also include the appropriate means for performing the amplification, in particular the PCR or RT-PCR, such as the polymerase and the necessary reagents including the suitable buffers, the nucleotides and the like.

The kits may also comprise beads or solid supports bearing the second members of the affinity binding pair. In a preferred embodiment, avidins or streptavidins are linked to beads or solid supports.

The kits may also enclose one or several of the necessary restriction enzymes to apply the method, in particular the first, second, third and fourth restriction enzymes. Preferably, the kit may include at least the first and the second restriction enzymes. In addition, the kit may include the third restriction enzyme. It can further include the fourth restriction enzyme.

All the particular features detailed for the method disclosed herein can also be contemplated for the kits.

The DNA to be treated may be also a set of a mixture of several nucleic acid sequence of different sequences. The nucleic acid can be a set of genes or a set of cDNA, or a chromosomal region, or whole genome. This nucleic acid mixture is cleaved. Preferably, the cleavage is performed by used a sequence independent technique of cleavage (SITC), such as sonication. Preferably, ends of nucleic acid molecules are repaired after each step of cleavage or of digestion and/or each step of circularization. Repairing may comprise restoring ends and/or phosphorylating ends. Restoration and phosphorylation may be performed by any method known by the skilled person. For example, restoration may be performed by using a specific DNA polymerase, such as T4 DNA polymerase, in presence of dNTP, and phosphorylation may be performed by using a DNA kinase, such as T4 polynucleotide kinase, in presence of ATP. The set of a mixture of several nucleic acid sequence is tailed by adding nucleotide to the DNA ends by Terminal transferase that catalyzes the addition of nucleotides, preferably ddNTP, to the 3′ terminus of DNA, preferably ddTTP.

The tailed set of a mixture of several nucleic acid sequence with ddTTP is ligated with T4 DNAligase in presence of ATP with a tailed of designed linear nucleic acid molecules of double-stranded polynucleotide here after.

In one embodiment, a designed linear nucleic acid molecules of double-stranded polynucleotide whose sequence is composed in the 5′ to 3′ direction
a reverse priming site,
a forward priming site,
a barcode sequence specific of each set of nucleic acid molecules, which is located between the forward priming site and the sequence from the set of a mixture of several nucleic acid sequence or between the sequence from the set of a mixture of several nucleic acid sequence and the reverse priming site, and
two recognition sites for two different restriction enzymes:
   a first recognition site for a first restriction enzyme which is located between the barcode sequence and the sequence from the set of a mixture of several nucleic acid sequence, and
   a second recognition site for a second restriction enzyme which is located between the reverse priming site and the forward priming site.

The designed linear nucleic acid molecules of double-stranded polynucleotide is tailed by adding nucleotide to the DNA ends by Terminal transferase that catalyzes the addition of nucleotides to the 3′ terminus of DNA, preferably ddATP.

One barcode is to be associated with each sequence of the set of a mixture of several nucleic acid sequence of “n” different sequences, “n” is comprised from 1 to 1000. Then, a library can be prepared from the association of one barcode to each sequence of the set of a mixture of several nucleic acid sequence by the method disclosed herein, thereby providing a library of fragments of the set of a mixture of several nucleic acid sequence associated to the same barcode.

In one embodiment, linear nucleic acid molecules provided in step a) comprise the sequence of each sequence of one set of a mixture of nucleic acid sequence at one of its end (e.g., FIG. 1a, FIG. 1d, FIG. 1g and FIG. 1h) and the method further comprises a step a′) of cleaving the nucleic acid molecules, after step a) and before step b), thereby providing a truncated sequence of each sequence of one set of a mixture of nucleic acid sequence. Preferably, the cleavage is performed by used a sequence independent technique of cleavage (SITC), such as sonication. In particular, if the sequence of each sequence of one set of a mixture of nucleic acid sequence is at the 3′ end of the linear nucleic acid molecules provided in step a) (e.g., FIG. 1a and FIG. 1h), the obtained nucleic acid molecules present a 3′ truncated sequence of each sequence of one set of a mixture of nucleic acid sequence. Alternatively, if the sequence of each sequence of one set of a mixture of nucleic acid sequence is at the 5′ end of the linear nucleic acid molecules provided in step a) (e.g., FIG. 1d and FIG. 1g), the obtained nucleic acid molecules present a 5′ truncated sequence of each sequence of one set of a mixture of nucleic acid sequence. Cleaved molecules may be separated by electrophoresis and molecules of the desired size may be purified from the electrophoresis gel.

In another embodiment, linear nucleic acid molecules provided in step a) do not comprise the sequence of each sequence of one set of a mixture of nucleic acid sequence at one of its end but comprise a recognition site for a fourth restriction enzyme located at the end of the sequence of each sequence of one set of a mixture of nucleic acid sequence that is opposite to the end adjacent to the barcode. In this embodiment, the method further comprises after step e),

f) digesting circular nucleic acid molecules obtained from step e) with the fourth restriction enzyme, thereby providing linear nucleic acid molecules comprising the non truncated end of the sequence of each sequence of one set of a mixture of nucleic acid sequence at one of its end;

g) cleaving digested nucleic acid molecules obtained from step f) in the sequence of each sequence of one set of a mixture of nucleic acid sequence, thereby providing linear nucleic acid molecules comprising a sequence of each sequence of one set of a mixture of nucleic acid sequence truncated in 5′ and 3′.

Cleaved molecules may be separated by electrophoresis and molecules of the desired size may be purified from the electrophoresis gel.

The method of invention may further comprise a step h) of circularizing nucleic acid molecules obtained from step g) by intra-molecular ligation. This ligation may be performed as disclosed above.

According to the structure of the nucleic acid molecules providing in step a), the circular molecules obtained from step h) comprise, in the 5′ to 3′ direction
a forward priming site, a barcode sequence, a sequence of each sequence of one set of a mixture of nucleic acid sequence truncated in 3′ and 5′, a recognition site for a third restriction enzyme, a reverse priming site and a curvature module comprising a recognition site for a second restriction enzyme, or
a forward priming site, a sequence of each sequence of one set of a mixture of nucleic acid sequence truncated in 5′ and in 3′, a barcode sequence, a reverse priming site and a curvature module comprising a recognition site for a second restriction enzyme, or
a forward priming site, a sequence of interest truncated in 5′ and in 3′, a barcode sequence, a reverse priming site and a curvature module comprising a recognition site for a second restriction enzyme.

In an embodiment, the nucleic acid molecules provided in step a) are attached to a first member of an affinity binding pair and the method further comprises one or several steps of binding nucleic acid molecules on a solid support through the interaction between the first member of an affinity binding pair attached to said nucleic acid molecules and second members of said affinity binding pair linked to the solid support. Useful solid supports include any rigid or semi-rigid surface on which a member of an affinity binding pair may be linked. The support can be any porous or non-porous water insoluble material, including, without limitation, membranes, filters, chips, magnetic or nonmagnetic uniform beads, and polymers. Preferably, the solid support is selected from silica-based membranes and beads. More preferably, the solid support is beads and more particularly polystyrene beads. These beads may have a diameter from 1 to 10 micrometer. In a particular embodiment, the nucleic acid molecules provided in step a) are attached to biotin and are bound to a solid support, preferably magnetic beads, coated with streptavidin. Nucleic acid molecules may be bound to a solid support before a circularization step by intra-molecular ligation in order to prevent intermolecular events or before each change of reaction mix in order to facilitate the recovering of nucleic acid molecules. The amount of solid support may be easily adjusted by the skilled person according to the concentration of nucleic acid molecules.

In a preferred embodiment, linear nucleic acid molecules obtained from step g) are bound to a solid support before circularization of step h).

In another embodiment, circular nucleic acid molecules obtained from step h) are then bound to a solid support.

Circular nucleic acid molecules obtained from step h) may be digested by the second restriction enzyme, thereby providing linear nucleic acid molecules comprising from 5′ end to 3′ end,
a forward priming site, a barcode sequence, a sequence of interest truncated in 5′ and 3′ and a reverse priming sequence, or
a forward priming site, a sequence of interest truncated in 5′ and 3′, a barcode sequence and a reverse priming sequence.

If the nucleic acid molecules comprise a curvature module comprising the recognition site for the second restriction enzyme, nucleic acid molecules obtained by digestion with the second restriction enzyme comprises at each end, a part of the curvature module.

Preferably, nucleic acid molecules are bound on a solid support prior to be digested with the second restriction enzyme.

Nucleic acid molecules digested with the second restriction enzyme may then be amplified, for example by PCR or by any other method known by the skilled person. Preferably, primers used for this amplification comprise:
a forward primer hybridizing to the forward priming site, and
a reverse primer hybridizing to the reverse priming site.

The product of this amplification may then be sequenced by using any high-throughput sequencing plateform.

### REFERENCES

Barany (1991) PCR Methods Appl, 1, 5-16;
Blanco et al. (1989) J Biol Chem, 264, 8935-8940;
Birkenmeyer (1985) Nucleic Acids Res, 13, 7107-7118;
Cahill et al. (1991) Clin Chem, 37, 1482-1485;
Chetverin and Spirin (1995) Prog Nucleic Acid Res Mol Biol, 51, 225-270;
Collins and Weissman (1984) Proc Natl Acad Sci USA 81, 6812-6816
Compton, J. (1991) Nature, 350, 91-92;
Fahy et al. (1991) PCR Methods Appl, 1, 25-33;
Fullwood et al., Genome Res. 2009. 19: 521-532;
Katanaev et al. (1995) Febs Lett, 359, 89-92;
Kitchin et al., (1986) J Biol Chem, 25, 11302-11309;
Landegren et al. (1988) Science, 241, 1077-1080;
Lizardi et al. (1998) Nat Genet, 19, 225-232;
Mamanova et al., (2010) Nature Method, 7, 111-118;
Notomi et al. (2000) Nucleic Acids Res, 28, E63;
Ulanovsky et al. (1986) PNAS, 83, 862-866;
Walker et al. (1992) Nucleic Acids Res, 20, 1691-1696;

In summary,

The method of invention relates to a new method for preparing a nucleic acid library, preferably a DNA library, to be sequenced, wherein said method comprises:

a) providing a set or a plurality of sets of linear nucleic acid molecules, each nucleic acid molecule comprising in its circularized form and in the 5′ to 3′ direction
a sequence of interest to be entirely or partially sequenced
a reverse priming site,
a forward priming site,
a barcode sequence specific of each set of nucleic acid molecules, which is located between the forward priming site and the sequence of interest or between the sequence of interest and the reverse priming site, and
two recognition sites for two different restriction enzymes:
   a first recognition site for a first restriction enzyme which is located between the barcode sequence and the sequence of interest, and
   a second recognition site for a second restriction enzyme which is located between the reverse priming site and the forward priming site;

b) circularizing said linear nucleic acid molecules by intra-molecular ligation;

c) digesting circularized nucleic acid molecules obtained from step b) with the first restriction enzyme;

d) cleaving digested nucleic acid molecules obtained from step c) in the sequence of interest; and

e) circularizing said cleaved nucleic acid molecules obtained from step d) by intra-molecular ligation,

thereby providing circularized nucleic acid molecules comprising, in the 5′ to 3′ direction, a truncated sequence of interest, a reverse priming site, a forward priming site and a barcode sequence which is between the forward priming site and the sequence of interest or between the sequence of interest and the reverse priming site.

According to the method, wherein the linear nucleic acid molecules provided in step a) comprise in their circularized form and in the 5′ to 3′ direction, a sequence of interest, a reverse priming site, a forward priming site and a barcode sequence located between the forward priming site and the sequence of interest.

According to the method, wherein the linear nucleic acid molecules provided in step a) further comprise a recognition site for a third restriction enzyme located upstream to the reverse priming site, said enzyme having a cleavage site upstream to said recognition site in the sequence of interest, and wherein the cleavage of step d) of claim 1 is achieved by digestion with said third restriction enzyme.

According to the method, wherein nucleic acid molecules provided in step a) further comprise a recognition site for a fourth restriction enzyme located upstream to the reverse priming site and wherein the method further comprises after step e):

f) digesting circularized nucleic acid molecules obtained from step e) with the fourth restriction enzyme having a cleavage site located between the reverse priming site and the sequence of interest in said circularized molecules of step e); and

g) cleaving digested nucleic acid molecules obtained from step f) in the sequence of interest,

thereby providing linear nucleic acid molecules comprising from 5′ end to 3′ end, a reverse priming site, a forward priming site, a barcode sequence and a sequence of interest truncated in 3′.

According to the method, wherein the linear nucleic acid molecules provided in step a) comprise from 5′ end to 3′ end, a reverse priming site, a forward priming site, a barcode sequence and a sequence of interest.

According to the method, wherein the method further comprises, after step a) and before step b), the step of cleaving linear nucleic acid molecules provided in step a), thereby providing linear nucleic acid molecules comprising from 5′ end to 3′ end, a reverse priming site, a forward priming site, a barcode sequence and a sequence of interest truncated in 3′.

According to the method, wherein the linear nucleic acid molecules provided in step a) comprise in their circularized form and in the 5′ to 3′ direction, a reverse priming site, a forward priming site, a sequence of interest and a barcode sequence located between the sequence of interest and the reverse priming site.

According to the method, wherein the linear nucleic acid molecules provided in step a) further comprise a recognition site for a third restriction enzyme located downstream to the forward priming site, said enzyme having a cleavage site downstream to said recognition site in the sequence of interest, and wherein the cleavage of step d) of claim 1 is achieved by digestion with said third restriction enzyme.

According to the method, wherein nucleic acid molecules provided in step a) further comprise a recognition site for a fourth restriction enzyme located downstream to the forward priming site, and wherein the method further comprises after step e):

f) digesting circularized nucleic acid molecules obtained from step e) with the fourth restriction enzyme having a cleavage site located between the forward priming site and the sequence of interest in said circularized molecules; and

g) cleaving digested nucleic acid molecules obtained from step f) in the sequence of interest,

thereby providing linear nucleic acid molecules comprising from 5′ end to 3′ end, a sequence of interest truncated in 5′, a barcode sequence, a reverse priming site and a forward priming site.

According to the method, wherein the linear nucleic acid molecules provided in step a) comprise from 5′ end to 3′ end, a sequence of interest, a barcode sequence, a reverse priming site and a forward priming site.

According to the method, wherein the method further comprises, after step a) and before step b), the step of cleaving linear nucleic acid molecules provided in step a), thereby providing linear nucleic acid molecules comprising from 5′ end to 3′ end, a sequence of interest truncated in 5′, a barcode sequence, a reverse priming site and a forward priming site.

According to the method, wherein the method further comprises the step h) of circularizing nucleic acid molecules obtained from step g) by intra-molecular ligation.

According to the method, wherein the nucleic acid molecules provided in step a) comprise a binding site for a first member of an affinity binding pair or is attached to a first member of an affinity binding pair.

The method according to the method, wherein the method further comprises one or several steps of binding nucleic acid molecules on a solid support through the interaction between the first member of an affinity binding pair attached to said nucleic acid molecules and second members of said affinity binding pair linked to the solid support, in particular before a circularizing step.

According to the method, wherein the method further comprises the step of binding nucleic acid molecules obtained from step g) before to perform the step h) of circularizing.

According to the method, wherein the method further comprises the step of digesting circularized nucleic acid molecules with the second restriction enzyme, thereby providing linear nucleic acid molecules comprising from 5′ end to 3′ end, a forward priming site, a truncated sequence of interest, a reverse priming site and a barcode sequence which is between the forward priming site and the sequence of interest or between the sequence of interest and the reverse priming site.

According to the method, wherein the method further comprises the step of amplifying barcode sequences and truncated sequences of interest from said linear nucleic acid molecules by using a pair of primers hybridizing on reverse and forward priming site.

According to the method, wherein the steps of cleaving nucleic acid molecules are performed by using a sequence independent technique of cleavage.

According to the method, wherein the sequence independent technique of cleavage is sonication.

According to the method, wherein the third restriction enzyme is selected from the group consisting of EcoP15I, MmeI, NmeAII, AcuI, BbvI, BceAI, BpmI, BpuEI, BseRI, BsgI, BsmFI, BtgZI, EciI, FokI, HgaI, I-CeuI, I-SceI, PI-PspI and PI-Scel.

According to the method, wherein the linear nucleic acid molecules provided in step a) further comprise a sequence of at least 15 base pairs which have less than 80% identity with the genome of the sequence of interest and which is located between the sequence of interest and the element upstream to said sequence of interest or between the sequence of interest and the element downstream to said sequence of interest.

According to the method, wherein the linear nucleic acid molecules provided in step a) further comprise a curvature module which is located, in their circularized form, between the reverse priming site and the forward priming site.

According to the method, wherein said curvature module comprises a binding site for a first member of an affinity binding pair or is attached to the first member of an affinity binding pair.

According to the method, wherein said curvature module comprises or consists of the sequence selected from the group consisting of sequences of SEQ ID No. 1 and SEQ ID No. 2.

According to the method, wherein the linear nucleic acid molecules provided in step a) have been obtained by one or several DNA amplification reactions

According to the method, wherein said DNA amplification reactions are followed by the step of degrading excess single-stranded primers.

According to the method, wherein the method further comprises the step of restoring and/or phosphorylating each ends of nucleic acid molecules after a step of cleavage and/or before a step of circularization.

According to the method, wherein the method further comprises the step of degrading non-circularized nucleic acid molecules with an endonuclease specific of linear nucleic acid molecules after a step of circularization.

According to the method, wherein nucleic acid molecules obtained from step h) have a length of 156 bp or a length of 156 bp plus a multiple of 21 bp.

According to the method, wherein a plurality of sets of linear nucleic acid molecules is provided in step a).

Nucleic acid library obtained from the method according to any one of the steps of the method.

A kit comprising at least one first forward primer comprising, from its 5′ end to its 3′ end,
a reverse priming site, a recognition site for a second restriction enzyme, and a forward priming site or a part thereof including its 5′ end; or
a reverse priming site, a curvature module comprising a recognition site for the second restriction enzyme, and a forward priming site or a part thereof including its 5′ end.

The kit according to the method, wherein the kit further comprises at least one second forward primer comprising, from its 5′ end to its 3′ end, a forward priming site or a part thereof including its 3′ end and overlapping the part of forward priming site of the first forward primer, a barcode sequence, a recognition site for a first restriction enzyme and either at least 10 nucleotides of the 5′ end of the sequence of interest or a universal priming site.

The kit according to the method, wherein the kit further comprises at least one second forward primer comprising, from its 5′ end to its 3′ end, a forward priming site or a part thereof including its 3′ end and overlapping the part of forward priming site of the first forward primer, a barcode sequence, a recognition site for a first restriction enzyme and a universal priming site.

The kit according to the method, wherein the kit comprises one first forward primer and several second forward primers differing by their barcode sequences and, if present, by the at least 10 nucleotides of the 5′ end of the sequence of interest.

The kit according to the method, wherein the at least one first forward primer further comprises, at its 3′ end, a barcode sequence, a recognition site for a first restriction enzyme and either at least 10 nucleotides of the 5′ end of the sequence of interest or a universal priming site.

The kit according to the method, wherein the at least one first forward primer further comprises, at its 3′ end, a barcode sequence, a recognition site for a first restriction enzyme and a universal priming site.

The kit according to the method, wherein the at least one first forward primer further comprises, at its 5′ end, recognition site for the third restriction enzyme and/or a recognition site for the fourth restriction enzyme.

The kit according to the method, wherein the kit comprises several first forward primers differing by their barcode sequences and, if present, by the at least 10 nucleotides of the 5′ end of the sequence of interest.

The kit according to the method, wherein the kit further comprises one reverse primer comprising at least 10 nucleotides of the 3′ end of the sequence of interest or several reverse primers, each comprising at least 10 nucleotides of the 3′ end of a different sequence of interest.

The kit according to the method, wherein the at least one first forward primer further comprises at its 3′ end a recognition site for the third restriction enzyme and/or a recognition site for the fourth restriction enzyme, and either at least 10 nucleotides of the 5′ end of the sequence of interest or a universal priming site.

The kit according to the method, wherein the at least one first forward primer further comprises at its 3′ end a recognition site for the third restriction enzyme and/or a recognition site for the fourth restriction enzyme, and a universal priming site.

The kit according to the method, wherein the kit comprises one first forward primer.

The kit according to the method, wherein the kit further comprises a reverse primer comprising, from its 5′ end to its 3′ end, a barcode sequence, a recognition site for a first restriction enzyme and at least 10 nucleotides of the 3′ end of the sequence of interest.

The kit according to the method, wherein the kit further comprises several reverse primers, differing by their barcode sequences and by the at least 10 nucleotides of the 3′ end of the sequence of interest.

The kit according to the method, wherein the kit further comprises at least one forward primer comprising, from its 5′ end to its 3′ end, a universal priming site and at least 10 nucleotides of the 5′ end of the sequence of interest.

The kit according to the method, wherein the kit further comprises several forward primers comprising, from its 5′ end to its 3′ end, a universal priming site and at least 10 nucleotides of the 5′ end of one of the different sequences of interest.

A kit comprising at least one first reverse primer comprising, from its 5′ end to its 3′ end,
a forward priming site, a recognition site for a second restriction enzyme, and a reverse priming site or a part thereof including its 3′ end; or
a forward priming site, a curvature module comprising a recognition site for the second restriction enzyme, and a reverse priming site or a part thereof including its 3′ end.

The kit according to the method, wherein the kit further comprises at least one second reverse primer comprising, from its 5′ end to its 3′ end, a reverse priming site or a part thereof including its 5′ end and overlapping the part of reverse priming site of the first reverse primer, a barcode sequence, a recognition site for a first restriction enzyme and either at least 10 nucleotides of the 3′ end of the sequence of interest or a universal priming site.

The kit according to the method, wherein the kit further comprises at least one second reverse primer comprising, from its 5′ end to its 3′ end, a reverse priming site or a part thereof including its 5′ end and overlapping the part of reverse priming site of the first reverse primer, a barcode sequence, a recognition site for a first restriction enzyme and a universal priming site.

The kit according to the method, wherein the kit comprises one first reverse primer and several second reverse primers differing by their barcode sequences and, if present, by the at least 10 nucleotides of the 3′ end of the sequence of interest.

The kit according to the method, wherein the at least one first reverse primer further comprises, at its 3′ end, a barcode sequence, a recognition site for a first restriction enzyme and either at least 10 nucleotides of the 3′ end of the sequence of interest or a universal priming site.

The kit according to the method, wherein the at least one first reverse primer further comprises, at its 3′ end, a barcode sequence, a recognition site for a first restriction enzyme and a universal priming site.

The kit according to the method, wherein the at least one first reverse primer further comprises, at its 5′ end, recognition site for the third restriction enzyme and/or a recognition site for the fourth restriction enzyme.

The kit according to the method, wherein the kit comprises several first reverse primers differing by their barcode sequences and, if present, by the at least 10 nucleotides of the 3′ end of the sequence of interest.

The kit according to the method, wherein the kit further comprise one forward primer comprising at least 10 nucleotides of the 5′ end of the sequence of interest or several forward primers, each comprising at least 10 nucleotides of the 5′ end of a different sequence of interest.

The kit according to the method, wherein the at least one first reverse primer further comprises at its 3′ end a recognition site for the third restriction enzyme and/or a recognition site for the fourth restriction enzyme, and either at least 10 nucleotides of the 3′ end of the sequence of interest or a universal priming site.

The kit according to the method, wherein the at least one first reverse primer further comprises at its 3′ end a recognition site for the third restriction enzyme and/or a recognition site for the fourth restriction enzyme, and a universal priming site.

The kit according to the method, wherein the kit comprises one first reverse primer.

The kit according to the method, wherein the kit further comprises a forward primer comprising, from its 5′ end to its 3′ end, a barcode sequence, a recognition site for a first restriction enzyme and at least 10 nucleotides of the 5′ end of the sequence of interest.

The kit according to the method, wherein the kit further comprises several forward primers, differing by their barcode sequences and by the at least 10 nucleotides of the 5′ end of the sequence of interest.

The kit according to the method, wherein the kit further comprises at least one reverse primer comprising, from its 5′ end to its 3′ end, a universal priming site and at least 10 nucleotides of the 3′ end of the sequence of interest.

The kit according to the method, wherein the kit further comprises several reverse primers comprising, from its 5′ end to its 3′ end, a universal priming site and at least 10 nucleotides of the 3′ end of one of the different sequences of interest.

The kit according to the method, wherein the first forward primer or first reverse primer comprises a curvature module comprising a recognition site for the second restriction enzyme.

The kit according to the method, wherein the curvature module is attached to a first member of an affinity binding pair, preferably biotin.

The kit according to the method, wherein the curvature module comprises or consists of the sequence selected from the group consisting of sequences of SEQ ID No. 1 and SEQ ID No. 2.

The kit according to the method, wherein the kit further comprises beads or solid supports bearing the second members of the affinity binding pair, preferably avidin or streptavidins.

The kit according to the method, wherein the kit further comprises one or several restriction enzymes selected from the group consisting of the first, second, third and fourth restriction enzymes.

A method for preparing a nucleic acid library, preferably a DNA library, to be sequenced, wherein said method comprises:
The DNA to be treated is a set of a mixture of several nucleic acid sequence of different sequences, is cleaved by using a sequence independent technique of cleavage, repaired and tailed by Terminal transferase at the 3′ terminus of DNA, preferably by ddTTP.
A designed linear nucleic acid molecules of double-stranded polynucleotide is tailed by Terminal transferase at the 3′ terminus of DNA, preferably by ddATP. The designed linear nucleic acid molecules of double-stranded polynucleotide sequence is composed in the 5′ to 3′ direction.

The tailed set of a mixture of several nucleic acid sequence with ddTTP is ligated with T4 DNA ligase in presence of ATP with a tailed of designed linear nucleic acid molecules of double-stranded polynucleotide.

a) providing a set or a plurality of sets of linear nucleic acid molecules, each nucleic acid molecule comprising in its circularized form and in the 5′ to 3′ direction
a reverse priming site,
a forward priming site,
a barcode sequence specific of each set of nucleic acid molecules, which is located between the forward priming site and the sequence from the set of a mixture of several nucleic acid sequence or between the sequence from the set of a mixture of several nucleic acid sequence and the reverse priming site, and
two recognition sites for two different restriction enzymes:
   a first recognition site for a first restriction enzyme which is located between the barcode sequence and the sequence from the set of a mixture of several nucleic acid sequence, and
   a second recognition site for a second restriction enzyme which is located between the reverse priming site and the forward priming site.

b) circularizing said linear nucleic acid molecules by intra-molecular ligation;

c) digesting circularized nucleic acid molecules obtained from step b) with the first restriction enzyme;

d) cleaving digested nucleic acid molecules obtained from step c) in the sequence of interest; and

e) circularizing said cleaved nucleic acid molecules obtained from step d) by intra-molecular ligation,

thereby providing circularized nucleic acid molecules comprising, in the 5′ to 3′ direction, a truncated sequence of interest, a reverse priming site, a forward priming site and a barcode sequence which is between the forward priming site and the sequence of interest or between the sequence of interest and the reverse priming site.

1. A method for preparing a nucleic acid library, preferably a DNA library, to be sequenced, wherein said method comprises:
a) providing a set or a plurality of sets of linear nucleic acid molecules, each nucleic acid molecule comprising in its circularized form and in the 5′ to 3′ direction
a sequence of interest to be entirely or partially sequenced
a reverse priming site,
a forward priming site,
a barcode sequence specific of each set of nucleic acid molecules, which is located between the forward priming site and the sequence of interest or between the sequence of interest and the reverse priming site, and
two recognition sites for two different restriction enzymes:
a first recognition site for a first restriction enzyme which is located between the barcode sequence and the sequence of interest, and
a second recognition site for a second restriction enzyme which is located between the reverse priming site and the forward priming site;
b) circularizing said linear nucleic acid molecules by intra-molecular ligation;
c) digesting circularized nucleic acid molecules obtained from step b) with the first restriction enzyme;
d) cleaving digested nucleic acid molecules obtained from step c) in the sequence of interest; and
e) circularizing said cleaved nucleic acid molecules obtained from step d) by intra-molecular ligation,
thereby providing circularized nucleic acid molecules comprising, in the 5′ to 3′ direction, a truncated sequence of interest, a reverse priming site, a forward priming site and a barcode sequence which is between the forward priming site and the sequence of interest or between the sequence of interest and the reverse priming site.

2. The method according to claim 1, wherein the linear nucleic acid molecules provided in step a) comprise in their circularized form and in the 5′ to 3′ direction, a sequence of interest, a reverse priming site, a forward priming site and a barcode sequence located between the forward priming site and the sequence of interest.

3. The method according to claim 2, wherein the linear nucleic acid molecules provided in step a) further comprise a recognition site for a third restriction enzyme located upstream to the reverse priming site, said enzyme having a cleavage site upstream to said recognition site in the sequence of interest, and wherein the cleavage of step d) of claim 1 is achieved by digestion with said third restriction enzyme.

4. The method according to claim 2 or 3, wherein nucleic acid molecules provided in step a) further comprise a recognition site for a fourth restriction enzyme located upstream to the reverse priming site and wherein the method further comprises after step e):
f) digesting circularized nucleic acid molecules obtained from step e) with the fourth restriction enzyme having a cleavage site located between the reverse priming site and the sequence of interest in said circularized molecules of step e); and
g) cleaving digested nucleic acid molecules obtained from step f) in the sequence of interest, thereby providing linear nucleic acid molecules comprising from 5′ end to 3′ end, a reverse priming site, a forward priming site, a barcode sequence and a sequence of interest truncated in 3′; and
h) optionally circularizing nucleic acid molecules obtained from step g) by intra-molecular ligation.

5. The method according to claim 2 or 3, wherein the linear nucleic acid molecules provided in step a) comprise from 5′ end to 3′ end, a reverse priming site, a forward priming site, a barcode sequence and a sequence of interest, and the method further comprises, after step a) and before step b), the step of cleaving linear nucleic acid molecules provided in step a), thereby providing linear nucleic acid molecules comprising from 5′ end to 3′ end, a reverse priming site, a forward priming site, a barcode sequence and a sequence of interest truncated in 3′.

6. The method according to claim 1, wherein the linear nucleic acid molecules provided in step a) comprise in their circularized form and in the 5′ to 3′ direction, a reverse priming site, a forward priming site, a sequence of interest and a barcode sequence located between the sequence of interest and the reverse priming site.

7. The method according to claim 6, wherein the linear nucleic acid molecules provided in step a) further comprise a recognition site for a third restriction enzyme located downstream to the forward priming site, said enzyme having a cleavage site downstream to said recognition site in the sequence of interest, and wherein the cleavage of step d) of claim 1 is achieved by digestion with said third restriction enzyme.

8. The method according to claim 7, wherein nucleic acid molecules provided in step a) further comprise a recognition site for a fourth restriction enzyme located downstream to the forward priming site, and wherein the method further comprises after step e):
f) digesting circularized nucleic acid molecules obtained from step e) with the fourth restriction enzyme having a cleavage site located between the forward priming site and the sequence of interest in said circularized molecules; and
g) cleaving digested nucleic acid molecules obtained from step f) in the sequence of interest, thereby providing linear nucleic acid molecules comprising from 5′ end to 3′ end, a sequence of interest truncated in 5′, a barcode sequence, a reverse priming site and a forward priming site; and
h) optionally circularizing nucleic acid molecules obtained from step g) by intra-molecular ligation.

9. The method according to claim 7, wherein the linear nucleic acid molecules provided in step a) comprise from 5′ end to 3′ end, a sequence of interest, a barcode sequence, a reverse priming site and a forward priming site, and the method further comprises, after step a) and before step b), the step of cleaving linear nucleic acid molecules provided in step a), thereby providing linear nucleic acid molecules comprising from 5′ end to 3′ end, a sequence of interest truncated in 5′, a barcode sequence, a reverse priming site and a forward priming site.

10. The method according to any claims 1 to 9, wherein the nucleic acid molecules provided in step a) comprise a binding site for a first member of an affinity binding pair or is attached to a first member of an affinity binding pair, and the method further comprises one or several steps of binding nucleic acid molecules on a solid support through the interaction between the first member of an affinity binding pair attached to said nucleic acid molecules and second members of said affinity binding pair linked to the solid support, in particular before a circularizing step.

11. The method according to any one of claims 1 to 10, wherein the method further comprises the step of digesting circularized nucleic acid molecules with the second restriction enzyme, thereby providing linear nucleic acid molecules comprising from 5′ end to 3′ end, a forward priming site, a truncated sequence of interest, a reverse priming site and a barcode sequence which is between the forward priming site and the sequence of interest or between the sequence of interest and the reverse priming site.

12. The method according to claim 11, wherein the method further comprises the step of amplifying barcode sequences and truncated sequences of interest from said linear nucleic acid molecules by using a pair of primers hybridizing on reverse and forward priming site.

13. The method according to any one of claims 1 to 12, wherein the steps of cleaving nucleic acid molecules are performed by using a sequence independent technique of cleavage, preferably by sonication.

14. The method according to any one of claims 1 to 13, wherein the linear nucleic acid molecules provided in step a) further comprise a universal priming site upstream or downstream to the sequence of interest.

15. The method according to any one of claims 1 to 14, wherein the linear nucleic acid molecules provided in step a) further comprise a curvature module which is located, in their circularized form, between the reverse priming site and the forward priming site, and which is attached to the first member of an affinity binding pair, preferably a curvature module comprising or consisting of the sequence selected from the group consisting of sequences of SEQ ID No. 1 and SEQ ID No. 2.

16. Nucleic acid library obtained from the method according to any one of claims 1 to 15.

17. A kit comprising at least one first forward primer comprising, from its 5′ end to its 3′ end,
a reverse priming site, a recognition site for a second restriction enzyme, and a forward priming site or a part thereof including its 5′ end; or
a reverse priming site, a curvature module comprising a recognition site for the second restriction enzyme, and a forward priming site or a part thereof including its 5′ end.

18. The kit according to claim 17, wherein the kit further comprises at least one second forward primer comprising, from its 5′ end to its 3′ end, a forward priming site or a part thereof including its 3′ end and overlapping the part of the forward priming site of the first forward primer, a barcode sequence, a recognition site for a first restriction enzyme and either at least 10 nucleotides of the 5′ end of the sequence of interest or a universal priming site.

19. The kit according to claim 17, wherein the at least one first forward primer further comprises, at its 3′ end, either
a barcode sequence, a recognition site for a first restriction enzyme and either at least 10 nucleotides of the 5′ end of the sequence of interest or a universal priming site, and optionally, at its 5′ end, a recognition site for the third restriction enzyme and/or a recognition site for the fourth restriction enzyme; or
a recognition site for the third restriction enzyme and/or a recognition site for the fourth restriction enzyme, and either at least 10 nucleotides of the 5′ end of the sequence of interest or a universal priming site.

20. A kit comprising at least one first reverse primer comprising, from its 5′ end to its 3′ end,
a forward priming site, a recognition site for a second restriction enzyme, and a reverse priming site or a part thereof including its 3′ end; or
a forward priming site, a curvature module comprising a recognition site for the second restriction enzyme, and a reverse priming site or a part thereof including its 3′ end.

21. The kit according to claim 20, wherein the kit further comprises at least one second reverse primer comprising, from its 5′ end to its 3′ end, a reverse priming site or a part thereof including its 5′ end and overlapping the part of the reverse priming site of the first reverse primer, a barcode sequence, a recognition site for a first restriction enzyme and either at least 10 nucleotides of the 3′ end of the sequence of interest or a universal priming site.

22. The kit according to claim 20, wherein the at least one first reverse primer further comprises, at its 3′ end, either
a barcode sequence, a recognition site for a first restriction enzyme and either at least 10 nucleotides of the 3′ end of the sequence of interest or a universal priming site and optionally, at its 5′ end, a recognition site for the third restriction enzyme and/or a recognition site for the fourth restriction enzyme; or
a recognition site for the third restriction enzyme and/or a recognition site for the fourth restriction enzyme, and either at least 10 nucleotides of the 3′ end of the sequence of interest or a universal priming site.

23. The kit according to any one of claims 17 to 22, wherein the first forward primer or first reverse primer comprises a curvature module comprising a recognition site for the second restriction enzyme and, attached to a first member of an affinity binding pair, such as a biotin, preferably a curvature module comprising or consisting of the sequence selected from the group consisting of SEQ ID No. 1 and SEQ ID No. 2.

24. A method for preparing a nucleic acid library, preferably a DNA library, to be sequenced, wherein said method comprises:
The DNA to be treated is a set of a mixture of several nucleic acid sequence of different sequences, is cleaved by using a sequence independent technique of cleavage, repaired and tailed by Terminal transferase at the 3′ terminus of DNA, preferably by ddTTP.
A designed linear nucleic acid molecules of double-stranded polynucleotide is tailed by Terminal transferase at the 3′ terminus of DNA, preferably by ddATP. The designed linear nucleic acid molecules of double-stranded polynucleotide sequence is composed in the 5′ to 3′ direction.
The tailed set of a mixture of several nucleic acid sequence with ddTTP is ligated with T4 DNAligase with a tailed of designed linear nucleic acid molecules of double-stranded polynucleotide.
a) providing a set or a plurality of sets of linear nucleic acid molecules, each nucleic acid molecule comprising in its circularized form and in the 5′ to 3′ direction
a reverse priming site,
a forward priming site,
a barcode sequence specific of each set of nucleic acid molecules, which is located between the forward priming site and the sequence from the set of a mixture of several nucleic acid sequence or between the sequence from the set of a mixture of several nucleic acid sequence and the reverse priming site, and
two recognition sites for two different restriction enzymes:
a first recognition site for a first restriction enzyme which is located between the barcode sequence and the sequence from the set of a mixture of several nucleic acid sequence, and
a second recognition site for a second restriction enzyme which is located between the reverse priming site and the forward priming site.
b) circularizing said linear nucleic acid molecules by intra-molecular ligation;
c) digesting circularized nucleic acid molecules obtained from step b) with the first restriction enzyme;
d) cleaving digested nucleic acid molecules obtained from step c) in the sequence of interest; and
e) circularizing said cleaved nucleic acid molecules obtained from step d) by intra-molecular ligation,
thereby providing circularized nucleic acid molecules comprising, in the 5′ to 3′ direction, a truncated sequence of interest, a reverse priming site, a forward priming site and a barcode sequence which is between the forward priming site and the sequence of interest or between the sequence of interest and the reverse priming site.

25. Nucleic acid library obtained from the method according to any one of claims 1 to 24.


SEQUENCE LISTING (2 sequences)

SEQ ID NO: 1 - DNA, 23 bp, Artificial Sequence
  Synthetic sequence derived from kinetoplast DNA minicircies of Crithidia fasciculata
ttaattaataataatttaattaa

SEQ ID NO: 2 - DNA, 37 bp, Artificial Sequence
  Synthetic sequence derived from kinetoplast DNA minicircies of Crithidia fasciculata
aatttccctgaattttaggttaattaacccccgaaaa